(19) United States
(12) Patent Application Publication
(10) Pub. No.: US 2003/0129146 A1
(43) Pub. Date: Jul. 10, 2003

(54) The use of bacterial phage associated lysing proteins for treating bacterial dental caries

(76) Inventors: Vincent Fischetti, West Hempstead, NY (US); Lawrence Loomis, Columbia, MD (US)

Correspondence Address: JONATHAN E. GRANT, 2120 L STREET, N.W., SUITE 210, WASHINGTON, DC, 20037, US

(21) Appl. No.: 10/219,250

(22) Filed: Aug. 16, 2002

Related U.S. Application Data
(63) Continuation-in-part of application No. 09/846,688, filed on May 2, 2001.
Application No. 09/846,688 is a continuation-in-part of application No. 09/497,495, filed on Apr. 18, 2000, now Pat. No. 6,238,661.
Application No. 09/497,495 is a continuation of application No. 09/395,636, filed on Sep. 14, 1999, now Pat. No. 6,056,954.
Application No. 09/395,636 is a continuation of application No. 08/962,523, filed on Oct. 31, 1997, now Pat. No. 5,997,862.

(51) Int. Cl. A61K 7/28; A61K 38/43

(52) U.S. Cl. 424/50

(57) ABSTRACT

A composition and method for treating bacterial dental caries by the use of an effective amount of at least one lytic specific for the bacteria causing caries. The lytic enzyme is genetically coded for by a bacteriophage which may be specific for said bacteria. The enzyme may be at least one lytic protein or peptides in a natural or modified form

The following application is a continuation-in-part of U.S. patent application Ser. No. 09/846,688, filed May 2, 2001, which is a continuation in part of 09/497,495, filed Apr. 18, 2000, now U.S. Pat. No. 6,238,661, which was a continuation of U.S. patent application Ser. No. 09/395,636, filed Sep. 14, 1999, now U.S. Pat. No. 6,056,954, issued May 2, 2000, which was a continuation in part of U.S. patent application Ser. No. 08/962,523, filed Oct. 31, 1997, now U.S. Pat. No. 5,997,862.

### I. BACKGROUND OF THE INVENTION

1. Field of the Invention

The present invention relates to methods and compositions for the treatment of bacterial infections by the use of bacteria-associated phage proteins, or peptides and peptide fragments thereof. More specifically, the invention pertains to phage lytic and/or holin proteins, or peptides and peptide fragments thereof, blended with a carrier for the treatment and prophylaxis of bacterial infection.

2. Description of the Prior Art

In the past, antibiotics have been used to treat various infections. The work of Selman Waksman in the introduction and production of Streptomycetes, and Dr. Fleming's discovery of penicillin, as well as the work of numerous others in the field of antibiotics, are well known. Over the years, there have been additions and chemical modifications to the “basic” antibiotics in attempts to make them more powerful, or to treat people allergic to these antibiotics.

Additionally, others have found new uses for these antibiotics. U.S. Pat. No. 5,260,292 (Robinson et al.) discloses the topical treatment of acne with aminopenicillins. The method and composition for topically treating acne and acneiform dermal disorders includes applying an amount of an antibiotic selected from the group consisting of ampicillin, amoxicillin, other aminopenicillins, and cephalosporins, and derivatives and analogs thereof, effective to treat the acne and acneiform dermal disorders. U.S. Pat. No. 5,409,917 (Robinson et al.) discloses the topical treatment of acne with cephalosporins.

However, as more antibiotics have been prescribed or used at an ever increasing rate for a variety of illnesses, increasing numbers of bacteria have developed a resistance to antibiotics. Larger doses of stronger antibiotics are now being used to treat ever more resistant strains of bacteria. Multiple antibiotic resistant bacteria have consequently developed. The use of more antibiotics and the number of bacteria showing resistance has led to increasing the amount of time that the antibiotics need to be used. Broad, nonspecific antibiotics, some of which have detrimental effects on the patient, are now being used more frequently. Also, antibiotics do not easily penetrate mucus linings.

Additionally, the number of people allergic to antibiotics appears to be increasing. Consequently, other efforts have been sought to first identify and then kill bacteria.

Attempts have been made to treat bacterial diseases with the use of bacteriophages. U.S. Pat. No. 5,688,501 (Merril, et al.) discloses a method for treating an infectious disease caused by bacteria in an animal with lytic or non-lytic bacteriophages that are specific for particular bacteria.

U.S. Pat. No. 4,957,686 (Norris) discloses a procedure of improved dental hygiene which comprises introducing into the mouth bacteriophages parasitic to bacteria which possess the property of readily adhering to the salivary pellicle.

It is to be noted that the direct introduction of bacteriophages into an animal to prevent or fight diseases has certain drawbacks. Typically, the bacteria should be in the right growth phase for the phage to attach. Both the bacteria and the phage should be in the correct and synchronized growth cycles. Additionally, there should be the right number of phages to attach to the bacteria; if there are too many or too few phages, there will be either no attachment or no production of the lysing enzyme. The phage should also be active enough. The phages are also inhibited by many things including bacterial debris from the organism it is going to attack. Further complicating the direct use of bacteriophages to treat bacterial infections is the possibility of immunological reactions, rendering the phage nonfunctional.

Consequently, others have explored the use of safer and more effective means to treat and prevent bacterial infections.

U.S. Pat. No. 5,604,109 (Fischetti et al.) relates to the rapid detection of Group A streptococci in clinical specimens, through the enzymatic digestion by a semi-purified Group C streptococcal phage associated lysin enzyme. The present invention is based upon the discovery that phage associated lytic enzymes specific for bacteria infected with a specific phage can effectively and efficiently break down the cell wall of the bacterium in question. At the same time, in most if not all cases, the semipurified enzyme is lacking in mammalian cell receptors and therefore tends to be less destructive to mammalian proteins and tissues when present during the digestion of the bacterial cell wall.

U.S. Pat. No. 5,985,271 (Fischetti, et. al.), U.S. Pat. No.5,997,862 (Fischetti et al.), and U.S. Pat. No. 6,017,528 (Fischetti et al.) disclose the compositions and their use in an oral delivery mode, such as a candy, chewing gum, lozenge, troche, tablet, a powder, an aerosol, a liquid or a liquid spray that contains a lysin enzyme produced by group C streptococcal bacteria infected with a C1 bacteriophage for the prophylactic and therapeutic treatment of Streptococcal A throat infections, commonly known as strep throat. This lysin enzyme is described in U.S. Pat. No. 5,604,109

The same general technique used to produce and purify a lysin enzyme shown in U.S. Pat. No. 5,604,109 may be used to manufacture other lytic enzymes produced by bacteria infected with a bacteriophage specific for that bacteria. Depending on the bacteria, there may be variations in the growth media and conditions.

The use of phage associated lytic enzymes produced by the infection of a bacteria with a bacteria specific phage has numerous advantages for the treatment of diseases. As the phage are targeted for specific bacteria, the lytic enzymes generally do not interfere with normal flora. Also, lytic phages primarily attack cell wall structures, which are not affected by plasmid variation. The actions of the lytic enzymes are fast and do not depend on bacterial growth. Additionally, lytic enzymes can be directed to the mucosal lining, where, in residence, they will be able to kill colonizing bacteria.

U.S. Pat. No. 6,056,954 (Fischetti et al.) discloses a method and composition for the prophylactic and/or therapeutic treatment of bacterial infections, comprising administering an effective amount of at least one lytic enzyme produced by a bacteria infected with a bacteriophage specific for the bacteria to the site of the infection. The lytic enzyme preferably comprises a carrier suitable for delivering the lytic enzyme to the site of the infection. This method and treatment may be used for treating upper respiratory infections, topical infections, vaginal infections, eye infections, ear infections, for parenteral treatment, and for most other bacterial infections.

U.S. Pat. No. 6,056,955 (Fischetti et al.) discloses the topical treatment of streptococcal infections.

U.S. Pat. No. 6,238,661 (Fischetti et al.) discloses compositions and methods for the prophylactic and therapeutic treatment of bacterial infections which comprise administering to an individual an effective amount of a composition comprising an effective amount of lytic enzyme and a carrier for delivering the lytic enzyme. This method and composition can be used for the treatment of upper respiratory infections, skin infections, wounds, burns, vaginal infections, eye infections, intestinal disorders and dental problems. The claims define the scope of the patent.

U.S. Pat. No. 6,248,324 (Fischetti et al.) discloses a method for treatment of bacterial infections of the digestive tract comprising administering a lytic enzyme specific for the infecting bacteria. The lytic enzyme is preferably in a carrier for delivering said lytic enzyme. The bacteria to be treated is selected from the group consisting of Listeria, Salmonella, E. coli, Campylobacter, and combinations thereof. The carrier for delivering at least one lytic enzyme to the digestive tract is selected from the group consisting of suppository enemas, syrups, or enteric coated pills.

U.S. Pat. No. 6,254,866 (Fischetti et al) discloses a method which comprises administering a lytic enzyme specific for the infecting bacteria. The lytic enzyme is preferably in a carrier for delivering said lytic enzyme. The bacteria to be treated is selected from the group consisting of Listeria, Salmonella, E. coli, Campylobacter, and combinations thereof. The carrier for delivering at least one lytic enzyme to the digestive tract is selected from the group consisting of suppository enemas, syrups, or enteric coated pills.

### SUMMARY OF THE INVENTION

The present invention arose from discoveries pertaining to the bacteriophage associated lytic proteins and holin proteins useful for destroying bacteria, including isozymes, analogs, and variants thereof in a natural or modified form either alone or in combination with complementary agents. The invention also features compositions that are site-specific for the mucosal membranes and pharmaceutically acceptable carriers for the treatment and amelioration of the infection of mucus membrane.

Accordingly, in one aspect, the present invention provides a pharmaceutical composition containing at least one bacteria-associated phage protein and peptides and peptide fragments thereof, isolated from one or more bacteria species, which phage proteins and peptides fragments thereof include phage lytic and/or holin proteins. In one embodiment, the lytic and/or holin proteins, including their isozymes, analogs, or variants, are used in a modified form. In another embodiment the lytic and/or holin proteins, including their isozymes, analogs, or variants, are used in a modified form or a combination of natural and modified forms. The modified forms of lytic and holin proteins are made synthetically by chemical synthesis and/or DNA recombinant techniques.

The invention features compositions containing at least one natural lytic protein, including isozymes, analogs, or variants thereof, isolated from the same or a different bacteria, with optional additions of a complementary agent.

According to one embodiment, the pharmaceutical composition includes one or more modified lytic protein, including isozymes, analogs, or variants thereof, produced by chemical synthesis or DNA recombinant techniques. In particular modified lytic protein is produced by chimerization , shuffling, or both. Preferably, the pharmaceutical composition contains combination of one or more natural lytic protein and one or more chimeric or shuffled lytic protein.

According to another embodiment of the invention, the pharmaceutical composition contains a peptide or a peptide fragment of at least one lytic protein derived from the same or different bacterial species, with an optional addition of one or more complementary agent, and a pharmaceutically acceptable carrier.

According to another embodiment of the invention, the pharmaceutical composition contains a peptide or a peptide fragment of at least one holin protein, or at least one holin and one lytic protein, which lytic and holin proteins are each derived from the same or different bacterial species, with an optional addition of a complementary agents, and a suitable carrier or diluent.

Also within the scope of the invention are compositions containing nucleic acid molecules that either alone or in combination with other nucleic acid molecules are capable of expressing an effective amount of lytic and/or holin proteins or a peptide fragment of the lytic and/or holin proteins in vivo. Also encompassed within the scope of this invention are cell cultures containing these nucleic acid molecules polynucleotides and vectors carrying and expressing these molecules in vitro or in vivo.

According to another embodiment of the invention, the pharmaceutical composition contains a complementary agent, including one or more conventional antibiotics.

According to another aspect of the invention, the pharmaceutical composition contains antibodies directed against a phage protein or peptide fragment of the invention.

According to another aspect, the invention provides, prevention, amelioration, or treatment of a variety of illnesses caused by Gram negative and/or Gram positive bacteria, including Streptococcal pyogenes, Streptococcal pneumoniae, Streptococcus fasciae, Hemophilus influenza, Listeria, Salmonella, E. coli, and Campylobacter.

The bacteria-phage associated proteins of this invention are administered to subjects in need thereof via several means of application. Means of application includes suitable carries that assist in delivery of the composition to the site of the infection and subsequent adsorption of the composition. The composition containing lytic and/or holin proteins or peptides and peptide fragments thereof, are incorporated into pharmaceutically acceptable carries and placed into appropriate means of application. Preferably, application means include suppository enemas, liquid means (for example, syrups, mouthwash, and eye drops in aqueous or nonaqueous form), solid means (for example, food stuff, confectionary, and toothpaste), bandages, tampons, topical creams, and inhalers, among others.

According to an embodiment of the invention, one or more phage proteins, or peptides and peptide fragments thereof, are placed in an inhaler to treat or prevent the spread of diseases localized in the mucus lining of the oral cavity and lungs. In a preferred embodiment, specific lytic proteins for tuberculosis are placed in a carrier and used to prevent or treat tuberculosis. In another embodiment, phage proteins are administered in the form of candy, chewing gum, lozenge, troche, tablet, a powder, aerosol, liquid spray, or toothpaste for the prevention or treatment of bacterial infections associated with upper respiratory tract illnesses.

According to another embodiment of invention, eye drops containing lytic proteins of Hemophilus, Pseudomonas, and/or Staphylococcus are used to directly treat eye infections.

In another embodiment of the invention, specific lytic proteins are used in the treatment of bacterial infections associated with topical or dermatological infections, administered in the form of a topical ointment or cream.

The invention also provides composition and method to treat burns and wounds by using one or more phage proteins, including preferably phage associated with Staphylococcus or Pseudomonas, incorporated into bandages to prevent or treat infections of burns and wounds.

According to another embodiment, lytic proteins, including those proteins or peptides and peptide fragments thereof specific for group B Streptococcus, are incorporated into tampons to prevent infection of the neonate during birth without disturbing normal vaginal flora so that women would not be overcome by yeast infection as a result of antibiotic therapy. Vaginal infections caused by Group B Streptococcus can cause premature birth and subsequent complications resulting in neonatal sepsis.

According to yet another embodiment of the invention, the pharmaceutical composition contains phage polypeptides, peptide fragments, nucleic acid molecules encoding phage protein or protein peptides fragments, antibody and antibody fragments, having biological activity either alone or with combination of other molecules polypeptides, peptides. In particular the phage polypeptides are selected from the group consisting of: a natural phage polypeptide, a naturally occurring allelic variant of said polypeptide, a modified polypeptide, and a polypeptide which is encoded by a nucleic acid molecule comprising a nucleotide sequence which is at least 65% identical to a nucleic acid encoding the said natural peptide. Additionally, the polypeptide of the invention is attached to heterologous amino acid sequences

According to another embodiment of the invention, phage peptides and peptide fragments thereof are antibodies that selectively bind to phage polypeptides.

The invention also features nucleic acid molecules as phage peptides and peptide fragments thereof The nucleic acid molecules of the invention are preferably attached to regulatory sequences and signal sequences, wherein said sequences affect site specificity and trans-membrane movements of said nucleic acid molecules. The signal sequences affect transportation of the nucleic acid molecules to the mucous membranes.

According to another aspect of the invention, a method for detecting the presence of a phage protein or peptides and peptide fragments thereof of the invention in a sample comprises: contacting the sample with a compound which selectively binds to said phage protein or peptides and peptide fragments thereof and determining whether the compound binds to said phage protein or peptides and peptide fragments thereof in said sample. In a preferred embodiment the compound is an antibody.

According to another aspect, a method for detecting the presence of a nucleic acid molecule of the invention is disclosed as comprising the steps of: contacting the sample with a nucleic acid probe or primer which selectively hybridizes to the nucleic acid molecule; and determining whether the nucleic acid probe or primer binds to a nucleic acid molecule in the sample.

According to another aspect of the invention, a kit is disclosed that contains a compound which selectively binds to a phage protein or peptides and peptide fragments thereof of the invention and instructions for use. In a preferred embodiment, a kit is disclosed that contains a compound which selectively hybridizes to a nucleic acid molecule of the invention and instructions for use.

According to another aspect, the invention discloses a drug screening method for identifying a compound which binds to a polypeptide of the invention comprising the steps of: contacting a polypeptide, or a cell expressing a polypeptide of the invention with a test compound; and determining whether the polypeptide binds to the test compound. The drug screening method also includes methods for modulating the activity of a polypeptide of the invention, as disclosed and described herein, comprising contacting a polypeptide or a cell expressing a polypeptide of the invention with a compound which binds to the polypeptide in a sufficient concentration to modulate the activity of the polypeptide.

### BRIEF DESCRIPTION OF THE DRAWINGS

FIG. 1 is an electron micrograph of group A streptococci treated with lysin showing the collapse of the cell wall and the cell contents pouring out;

FIG. 2 is a graph for the killing of S. pneumoniae (#DCC 1490) serotype 14 with PAL at various dilutions;

FIG. 3 is a graph showing the decrease of bacterial titer within 30 seconds after addition of 100 U Pal phage enzyme;

FIG. 4 is a series of graphs showing the decrease of the Bacterial titer with 30 seconds after the addition of 100, 1,000, and 10,000 U Pal lytic Enzyme;

FIG. 5 is a series of graphs showing the decrease of bacterial titer within 30 seconds after addition of different amounts of U Pal.

FIG. 6 depicts a histogram showing Group A Streptococci, Group B to N Streptococci, and oral Streptococci, with the optical density of different strains of bacteria at OD650/min. measured against different concentration of Pal enzyme; and

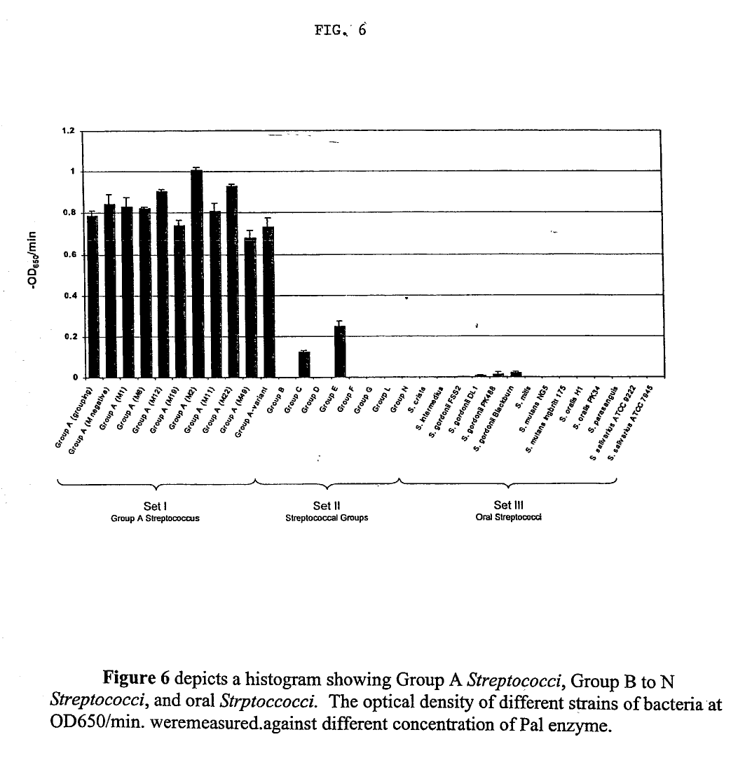

FIG. 7 shows polyacrylamide gel showing molecular weight of a lysin peptide.

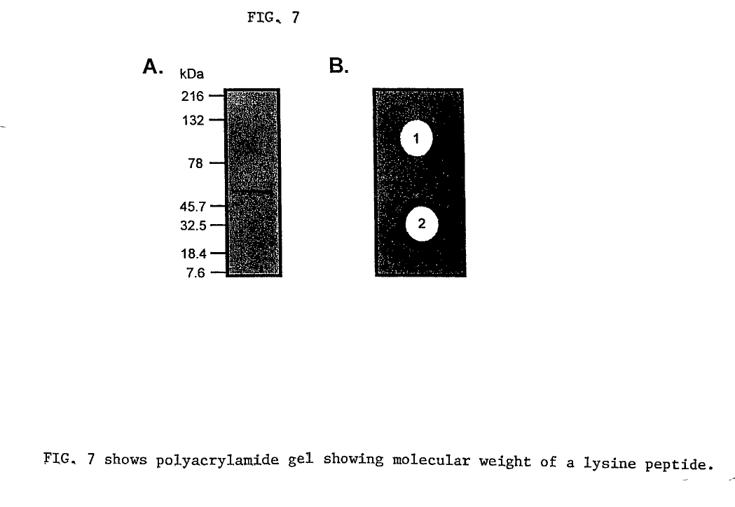

### IV. DETAILED DESCRIPTION OF THE INVENTION

This invention, as described and disclosed herein, features therapeutic compositions containing one or more bacteria-associated phage proteins or protein peptides fragments, including isozymes, analogs, or variants of phage enzymes or phage peptides and peptide fragments thereof in a natural or modified form. as active drugs and the method of use of such compositions in therapeutic, diagnostic, and drug screening.

The active drug of the invention, as described herein, includes one or more bacteria-associated phage proteins, peptides and peptide fragments thereof. Bacteria-associated phage proteins, as disclosed herein, include a variety of bacteria-specific phage lysin and holin proteins that are derived from one or several bacterial species.

Bacteriophage lytic proteins specifically cleave bonds that are present in the peptidoglycan of bacterial cells. Since the peptidoglycan is highly conserved among all bacteria, there are only a few bonds to be cleaved to disrupt the cell wall. Proteins that have the ability to hydrolyze components of a bacterial peptidoglycan fall into one of four categories:

1. N-acetylmuramoyl-L-alanine amidases (E.C. 3.5.1.28)—These proteins hydrolyze the link between N-acetylmuramoyl residues and L-amino acid residues in certain bacterial cell-wall glycopeptides.

Streptococcal lysin belongs to this family of lytic proteins. Of the 27 sequenced amidases, only the five highlighted are of bacteriophage origin. The rest are autolysins of bacterial origin.

2. Lysozyme. (EC 3.2.1.17), also known as muramidase. This protein hydrolyses the 1,4-beta-linkages between N-acetyl-D-glucosamine and N-acetylmuramic acid in peptidoglycan heteropolymers of the prokaryotes cell walls.

Of the 94 known sequences, 15 are encoded by bacteriophages,.

3. Beta 1,4 N-acetyl-D-glucosaminidase (EC 3.2.1.14), also known as Chitinase or Chitodextrinase. Hydrolysis of the 1 _{—}4-beta-linkages of N-acetyl-D-glucosamine polymers of chitin. These proteins are found primarily in the plant kingdom, although some are found in bacteria. None of the 104 known proteins are encoded by bacteriophages. However, many of these proteins that are produced by bacteria also possess lysozyme activity, and are usually classified with the other lysozymes.

4. Endopeptidase that cleaves the cross bridge of the peptidoglycan. The only known endopeptidase to be characterized extensively which acts on the peptidoglycan is lysostaphin (EC 3.4.24.75) This is a metalloprotease that hydrolyses the -Gly-1-Glybond in the pentaglycine inter-peptide link joining staphylococcal cell wall peptidoglycans. This protein is found several streptococcal species, but it is not encoded by bacteriophages. The only reported phage encoded endopeptidase that acts on the peptidoglycan is from a Pseudomonas phi 6 phage.

The majority of reported phage proteins are either muramidases or amidases. Fischetti et al (1974) reported that the C1 streptococcal phage lysine protein was an, amidase. Garcia et al (1987, 1990) reported that the CP-1 lysin from an S. pneumoniae phage was a muramidase. Caldentey and Bamford (1992) reported that a lytic protein from the phi 6 Pseudomonas phage was an endopeptidase, splitting the peptide bridge formed by meso-diaminopimilic acid and D-alanine. The E.coli T1 and T6 phage lytic proteins are amidases as is the lytic protein from Listeria phage (ply) (Loessner et al 1996).

Infection of the Hemophilus bacteria by Bacteriophage HP1 (a member of the P2-like phage family with strong similarities to coliphages P2 and 186, and some similarity to the retrophage Ec67) produces a lytic protein capable of lysing the bacteria. The lytic protein for Streptococcus pneumoniae, previously identified as a N-acetyl-muramoyl-L-alanine amidase, is produced by infecting Streptococcus pneumoniae with the Pal bacteriophage. The therapeutic composition contains either or both of the lytic proteins produced by these two bacteria, and also contains other lytic proteins from other bacteria.

These proteins are specifically effective in prophylactic and therapeutic treatment of bacterial infection of the upper respiratory tract. The infection can be prophylactically or therapeutically treated with a composition comprising an effective amount of at least one lytic protein produced by a bacteria being infected with a bacteriophage specific for that bacteria, and an application means for delivering the lytic protein to the cite of the infection, for example, mouth, throat, or nasal passage

For example, Streptococcus group A that produces what is commonly known as “strep” throat is treated prophylactically and therapeutically by the application of lytic proteins specific for Streptococcus group A. When group C Streptococci are infected with a C1 bacteriophage, a lytic protein is produced specific for the lysing of Streptococcus group A. The composition used for the prophylactic and therapeutic treatment of a strep infection includes, for example, one or more lytic proteins and a pharmaceutically acceptable carrier to the mucosal lining of the oral and nasal cavity, such that the protein reaches the mucosa lining.

There are a large number of phages which will attach to specific bacteria and produce proteins which will lyse that particular bacteria. The following are a list of bacteriophages and bacteria for which they are specific. It is noted that the bacteria and bacteriophages of the invention is not limited to the list disclosed below.

### BACTERIOPHAGES

There are a large number of phages which will attach to specific bacteria and produce enzymes which will lyse that particular bacteria. The following are a list of bacteriophages and bacteria for which they are specific:

Streptococci

Pseudomonas

Pneumococci

Salmonella

Staphylococci

Shigella

Haemophilus

Listeria

Mycobacteria

Vibrio

Corynebacteria

Bacillus

Spirochete

Myxococcus

Burkholderia

Brucella

Yersinia

Clostridium

Campylobacter

Neisseria

Actinomycetes

Agrobacterium

Alcaligenes

Clostridium

Coryneforms

Cyanobacteria

Enterobacteria

Lactobacillus

Lactoctococcus

Micrococcus

Pasteurella

Rhizobium

Xanthomonas

Bdellovibrio

mollicutes

Chlamydia

Spiroplasma

Caulobacter

Various phages which can be used to infect these bacteria and create the lytic enzyme include:

| BACTERIA | PHAGE(S) |
|---|---|
| Actinomycetes | A1-Dat, Bir, M1, MSP8, P-a-1, R1, R2, SV2, |
| | VP5, PhiC, ψ31C, ψUW21, ψ115-A, ψ50A, 119, |
| | SK1, 108/016 |
| Aeromonas | 29, 37, 43, 51, 59.1 |
| Altermonas | PM2 |
| Bacillus | AP50, ?ψNS11, BLE, Ipy-1, MP15, mor1, PBP1, |
| | SPP1, Spbb, type F, alpha, ψ105, 1A, II, Spy-2, |
| | SST, G, MP13, PBS1, SP3, SP8, SP10, SP15, |
| | SP50 |
| Bdellovibrio | MAC-1, MAC-1′, MAC-2, MAC-4, |
| | MAC-4′, MAC-5, MAC-7 |
| Caulobacter | ψpCb2, ψCb4, ψCb5, ψCb8r, ψCb9, ψCB12r, |
| | ψCb23r, ψCP2, ψCP18, ψCr14, ψCr28, PP7 |
| | ψCb2, ψCb4, ψCb5, ψCb8r, ψCb9, ψCB12r, |
| | ψCb23r, ψCP2, ψCP18, ψCr14, ψCr28, PP7 |
| Chlamydia | Chp-1 |
| Clostridium | F1, HM7, HM3, CEB, |
| Coliform | AE2, dA, Ec9, f1, fd, HR, M13, ZG/2, ZJ/2 |
| Coryneforms | Arp, BL3, CONX, MT, Beta, A8010, A19 |
| Cyanobacteria | S-2L, S-4L, N1, AS-1, S-6(L) |
| Enterobacter | C-2, If1, 1f2, Ike, I2-2, PR64FS, SF, tf-1, PRD1, |
| | H-19J, B6, B7, C-1, C2, Jersey, ZG/3A, TS, ViII, |
| | b4, chi, Beccles, tu, PRR1, 7s, C-1, c2, fcan, |
| | folac, Ialpha, M, pilhalpha, R23, R34, ZG/1, |
| | ZIK/1, ZJ/1, ZL/3, ZS/3, alpha15, f2, fr, FC3-9, |
| | K19, Mu, 01, P2, Vil, ψ92, 121, 16-19, 9266, |
| | C16, DdVI, PST, SMB, SMP2, al, 3, 3T+, 9/0, |
| | 11F, 50, 66F, 5845, 8893, M11, QB, ST, TW18, |
| | VK, FI, ID2, fr, f2, |
| Listeria | H387, 2389, 2671, 2685, 4211 |
| Micrococcus | N1, N5 |
| Mycobacterium | Lacticola, Leo, R1-Myb, 13 |
| Pasteurella | C-2, 32, AU |
| Pseudomonas | Phi6, Pf1, Pf2, Pf3, D3, Kf1, M6, PS4, SD1, |
| | PB-1, PP8, PS17, nKZ, nW-14, n1, 12S, |
| Staphyloccous | 3A, B11-M15, 77, 107, 187, 2848A, Twort |
| Streptococcus | A25, A25 PE1, A25 VD13, A25 omega8, A25 |
| | 24 |
| Steptococcus A | |
| Vibrio | OXN-52P, VP-3, VP5, VP11, alpha3alpha, IV, kappa, |
| 06N- | 22-P, VP1, x29, II, nt-1, |
| Xanthomonas | Cf, Cflt, Xf, Xf2, XP5 |

There are numerous other phages infecting these and other bacteria. The bacteriophages are normally grouped into family, genus and species, including Genus Chlamydiamicrovirus, Genus Bdellomicrovirus, Genus Spiromicrovirus, Genus Microvirus, Genus Microvirus, Genus Levivirus, Genus Allolevivirus, and other genuses

The composition of this invention contains phage peptides and peptide fragments thereof as well as, or instead of, phage proteins.

Phage proteins, as disclosed herein; include phage polypeptides, peptide fragments, nucleic acid molecules encoding phage protein or protein peptides fragments, antibody and antibody fragments, having biological activity either alone or with combination of other molecules

Nucleic acid molecules, as disclosed herein, include genes, gene fragments polynucleotides, oligonucleotides, DNA, RNA, DNA-RNA hybrids, EST, SNIPs. genomic DNA, cDNA, mRNA, antisense RNA, Ribozyme vectors containing nucleic acid molecules, regulatory sequences, and signal sequences. Nucleic acid molecules of this invention include any nucleic acid-based molecule that either alone or in combination with other molecules produce an oligonucleotide molecule capable or incapable of translation into a peptide.

Another example of a bacteria-associated phage protein used in the composition of this invention is the holin proteins. Holin proteins produce holes in the cell membrane. More specifically, holins form lethal membrane lesions that terminate cell respiration. Like the lytic proteins, holin proteins are coded for and carried by a phage. In fact, it is quite common for the genetic code of the holin protein to be next to or even within the code for the phage lytic protein. Most holin protein sequences are short, and overall, hydrophobic in nature, with a highly hydrophilic carboxy-terminal domain. In many cases, the putative holin protein is encoded on a different reading frame within the enzymatically active domain of the phage. In other cases, the holin protein is encoded on the DNA next or close to the DNA coding for the cell wall lytic protein. Holin proteins are frequently synthesized during the late stages of phage infection and are found in the cytoplasmic membrane where they cause membrane lesions

Holins can be grouped into two general classes based on primary structure analysis. Class I holins are usually 95 residues or longer and may have three potential transmembrane domains. Class II holins are usually smaller, at approximately 65-95 residues, with the distribution of charged and hydrophobic residues indicating two TM domains (Young, et al. Trends in Microbiology v. 8, No. 4, March 2000). At least for the phages of gram-positive hosts, however, the dual-component lysis system may not be universal. Although the presence of holins has been shown or suggested for several phages, no genes have yet been found encoding putative holins for all phages. Holins have been shown to be present in several bacteria, including, for example, lactococcal bacteriophage Tuc2009, lactococcal NLC3, pneumococcal bacteriophage EJ-1, Lactobacillus gasseri bacteriophage nadh, Staphylococcus aureus bacteriophage Twort, Listeria monocytogenes bacteriophages, pneumococcal phage Cp-1, Bacillus subtillis phage M29, Lactobacillus delbrueckki bacteriophage LL-H lysin, and bacteriophage N11 of Staphylococcus aureus. (Loessner, et al., Journal of Bacteriology, August 1999, p. 4452-4460).

The natural form of the protein or peptides fragments, as disclosed herein, includes an “isolated” or “purified” phage protein or peptides fragments, or a biologically active portion thereof that is substantially free of cellular material or other contaminating proteins from the cell or tissue source from which the protein is derived, or substantially free of chemical precursors or other chemicals when isolated. The language “substantially free of cellular material” includes preparations of a protein in which the protein is separated from cellular components of the host bacteria from which it is isolated. Thus, proteins or peptides and peptide fragments thereof that are substantially free of bacterial material include preparations of proteins or peptides and peptide fragments thereof having less than about 30%, 20%, 10%, or 5% (by dry weight) of heterologous protein (also referred to herein as a “contaminating protein”).

The modified form of the protein or peptides and peptide fragments, as disclosed herein, includes proteins or peptides and peptide fragments that are chemically synthesized or prepared by recombinant DNA techniques, or both. These techniques include, for example, chimerization and shuffling. When the protein or peptide is produced by chemical synthesis, it is preferably substantially free of chemical precursors or other chemicals, i.e., it is separated from chemical precursors or other chemicals which are involved in the synthesis of the protein. Accordingly, such preparations of the protein have less than about 30%, 20%, 10%, 5% (by dry weight) of chemical precursors or compounds other than the polypeptide of interest.

The invention also provides chimeric proteins or peptides fragments, which include fusion proteins. Chimeric proteins or peptides are produced, for example, by combining two or more proteins having two or more active sites. Chimeric proteins and peptides can act independently on the same or different molecules, and hence have a potential to treat two or more different bacterial infections at the same time. Chimeric proteins and peptides also are used to treat a bacterial infection by cleaving the cell wall in more than one location.

As used herein, a “chimeric protein” or “fusion protein” comprises all or (preferably a biologically active) part of a polypeptide of the invention operably linked to a heterologous polypeptide. The term “operably linked” means that the polypeptide of the invention and the heterologous polypeptide are fused in-frame. The heterologous polypeptide can be fused to the N-terminus or C-terminus of the polypeptide of the invention. Chimeric proteins are produced enzymatically by chemical synthesis, or by recombinant DNA technology.

One useful fusion protein is a GST fusion protein in which the polypeptide of the invention is fused to the C-terminus of a GST sequence. Such a chimeric protein can facilitate the purification of a recombinant polypeptide of the invention.

In another embodiment, the chimeric protein or peptide contains a heterologous signal sequence at its N-terminus. For example, the native signal sequence of a polypeptide of the invention can be removed and replaced with a signal sequence from another protein. For example, the gp67 secretory sequence of the baculovirus envelope protein can be used as a heterologous signal sequence (Current Protocols in Molecular Biology, Ausubel et al., eds., John Wiley & Sons, 1992). Other examples of eukaryotic heterologous signal sequences include the secretory sequences of melittin and human placental alkaline phosphatase (Stratagene; La Jolla, Calif.). In yet another example, useful prokaryotic heterologous signal sequences include the phoA secretory signal (Sambrook et al., supra) and the protein A secretory signal (Pharmacia Biotech; Piscataway, N.J.).

In yet another embodiment, the fusion protein is an immunoglobulin fusion protein in which all or part of a polypeptide of the invention is fused to sequences derived from a member of the immunoglobulin protein family. An immunoglobulin fusion protein of the invention can be incorporated into a pharmaceutical composition and administered to a subject to inhibit an interaction between a ligand (soluble or membrane-bound) and a protein on the surface of a cell (receptor), to thereby suppress signal transduction in vivo. The immunoglobulin fusion protein can alter bioavailability of a cognate ligand of a polypeptide of the invention. Inhibition of ligand/receptor interaction may be useful therapeutically, for both treating bacterial-associated diseases and disorders for modulating (i.e. promoting or inhibiting) cell survival. Moreover, an immunoglobulin fusion protein of the invention can be used as an immunogen to produce antibodies directed against a polypeptide of the invention in a subject, to purify ligands and in screening assays to identify molecules which inhibit the interaction of receptors with ligands.

Chimeric and fusion proteins and peptides of the invention can be produced by standard recombinant DNA techniques. In another embodiment, the fusion gene can be synthesized by conventional techniques, including automated DNA synthesizers. Alternatively, PCR amplification of gene fragments can be carried out using anchor primers which give rise to complementary overhangs between two consecutive gene fragments which subsequently can be annealed and reamplified to generate a chimeric gene sequence (see, i.e., Ausubel et al., supra). Moreover, many expression vectors are commercially available that already encode a fusion moiety (i.e., a GST polypeptide). A nucleic acid encoding a polypeptide of the invention can be cloned into such an expression vector such that the fusion moiety is linked in-frame to the polypeptide of the invention.

As used herein, shuffled proteins or peptides are molecules in which the genes, gene products, or peptides for more than one related phage protein or protein peptide fragments have been randomly cleaved and reassembled into a more active or specific protein. Shuffled oligonucleotides, peptides or peptide fragment molecules are selected or screened to identify a molecule having a desired functional property. This method is described, for example, in Stemmer, U.S. Pat. No. 6,132,970. (Method of shuffling polynucleotides); Kauffman, U.S. Pat. No. 5, 976,862 (Evolution via Condon-based Synthesis) and Huse, U.S. Pat. No. 5,808,022 (Direct Codon Synthesis). (The contents of these and all other patents and papers cited are incorporated herein by reference.)

Shuffling is used to create a protein that is 10 to 100 fold more active than the template protein. The template protein is selected among different varieties of lysin or holin proteins. The shuffled protein or peptides constitute, for example, one or more binding domains and one or more catalytic domains. Each binding or catalytic domain is derived from the same or a different phage or phage protein. The shuffled domains are either oligonucleotide based molecules, as gene or gene products, that either alone or in combination with other genes or gene products are translatable into a peptide fragment, or they are peptide-based molecules. Gene fragments include any molecules of DNA, RNA, DNA-RNA hybrid, antisense RNA, Ribozymes, ESTs, SNIPs and other oligonucleotide-based molecules that either alone or in combination with other molecules produce an oligonucleotide molecule capable or incapable of translation into a peptide.

In addition, libraries of fragments of the coding sequence of a polypeptide of the invention can be used to generate a variegated population of polypeptides for screening and subsequent selection of variants. For example, a library of coding sequence fragments can be generated by treating a double stranded PCR fragment of the coding sequence of interest with a nuclease under conditions wherein nicking occurs only about once per molecule, denaturing the double stranded DNA, renaturing the DNA to form a double stranded DNA which can include sense/antisense pairs from different nicked products, removing single stranded portions from reformed duplexes by treatment with S1 nuclease, and ligating the resulting fragment library into an expression vector. By this method, an expression library can be derived which encodes N-terminal and internal fragments of various sizes of the protein of interest.

Several techniques are known in the art for screening gene products of combinatorial libraries made by point mutations or truncation, and for screening cDNA libraries for gene products having a selected property. The most widely used techniques, which are amenable to high through-put analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a technique which enhances the frequency of functional mutants in the libraries, can be used in combination with the screening assays to identify variants of a protein of the invention (Arkin and Yourvan (1992) Proc. Natl. Acad. Sci. USA 89:7811-7815; Delgrave et al. (1993) Protein Engineering 6(3):327-331).

Biologically active portions of a protein or peptide fragment of the invention, as described herein, include polypeptides comprising amino acid sequences sufficiently identical to or derived from the amino acid sequence of the phage protein of the invention, which include fewer amino acids than the full length protein of the phage protein and exhibit at least one activity of the corresponding full-length protein. Typically, biologically active portions comprise a domain or motif with at least one activity of the corresponding protein. A biologically active portion of a protein or protein fragment of the invention can be a polypeptide which is, for example, 10, 25, 50, 100 less or more amino acids in length. Moreover, other biologically active portions, in which other regions of the protein are deleted, or added can be prepared by recombinant techniques and evaluated for one or more of the functional activities of the native form of a polypeptide of the invention.

A signal sequence of a polypeptide of the invention can facilitate transmembrane movement of the protein and peptides and peptide fragments of the invention to and from mucous membranes, as well as by facilitating secretion and isolation of the secreted protein or other proteins of interest. Signal sequences are typically characterized by a core of hydrophobic amino acids which are generally cleaved from the mature protein during secretion in one or more cleavage events. Such signal peptides contain processing sites that allow cleavage of the signal sequence from the mature proteins as they pass through the secretory pathway. Thus, the invention pertains to the described polypeptides having a signal sequence, as well as to the signal sequence itself and to the polypeptide in the absence of the signal sequence (i.e., the cleavage products).

In one embodiment, a nucleic acid sequence encoding a signal sequence of the invention can be operably linked in an expression vector to a protein of interest, such as a protein which is ordinarily not secreted or is otherwise difficult to isolate. The signal sequence directs secretion of the protein, such as from an eukaryotic host into which the expression vector is transformed, and the signal sequence is subsequently or concurrently cleaved. The protein can then be readily purified from the extracellular medium by art recognized methods. Alternatively, the signal sequence can be linked to a protein of interest using a sequence which facilitates purification, such as with a GST domain.

In another embodiment, a signal sequence of the present invention can be used to identify regulatory sequences, i.e., promoters, enhancers, repressors. Since signal sequences are the most amino-terminal sequences of a peptide, it is expected that the nucleic acids which flank the signal sequence on its amino-terminal side will be regulatory sequences that affect transcription. Thus, a nucleotide sequence which encodes all or a portion of a signal sequence can be used as a probe to identify and isolate the signal sequence and its flanking region, and this flanking region can be studied to identify regulatory elements therein.

The present invention also pertains to variants of the polypeptides of the invention. Such variants have an altered amino acid sequence which can function as either agonists (mimetics) or as antagonists. Variants can be generated by mutagenesis, i.e., discrete point mutation or truncation. An agonist can retain substantially the same, or a subset, of the biological activities of the naturally occurring form of the protein. An antagonist of a protein can inhibit one or more of the activities of the naturally occurring form of the protein by, for example, competitively binding to a downstream or upstream member of a cellular signaling cascade which includes the protein of interest. Thus, specific biological effects can be elicited by treatment with a variant of limited function. Treatment of a subject with a variant having a subset of the biological activities of the naturally occurring form of the protein can have fewer side effects in a subject relative to treatment with the naturally occurring form of the protein.

Variants of a protein of the invention which function as either agonists (mimetics) or as antagonists can be identified by screening combinatorial libraries of mutants, i.e., truncation mutants, of the protein of the invention for agonist or antagonist activity. In one embodiment, a variegated library of variants is generated by combinatorial mutagenesis at the nucleic acid level and is encoded by a variegated gene library. A variegated library of variants can be produced by, for example, enzymatically ligating a mixture of synthetic oligonucleotides into gene sequences such that a degenerate set of potential protein sequences is expressible as individual polypeptides, or alternatively, as a set of larger fusion proteins (i.e., for phage display). There are a variety of methods which can be used to produce libraries of potential variants of the polypeptides of the invention from a degenerate oligonucleotide sequence. Methods for synthesizing degenerate oligonucleotides are known in the art (see, i.e., Narang (1983) Tetrahedron 39:3; Itakura et al. (1984) Annu. Rev. Biochem. 53:323; Itakura et al. (1984) Science 198:1056; Ike et al. (1983) Nucleic Acid Res. 11:477).

A phage protein or peptide fragment of this invention can be used as an immunogen to generate antibodies using standard techniques for polyclonal and monoclonal antibody preparation. The full-length polypeptide or protein can be used or, alternatively, the invention provides antigenic peptide fragments for use as immunogens. The antigenic peptide of a protein of the invention comprises at least 8 (preferably 10, 15, 20, or 30) amino acid residues of the amino acid sequence of a phage protein or protein peptide fragments of the invention. and encompasses an epitope of the protein such that an antibody raised against the peptide forms a specific immune complex with the protein.

An immunogen typically is used to prepare antibodies by immunizing a suitable subject, (i.e., rabbit, goat, mouse or other mammal). An appropriate immunogenic preparation can contain, for example, recombinantly expressed or chemically synthesized polypeptide. The preparation can further include an adjuvant, such as Freund's complete or incomplete adjuvant, or similar immunostimulatory agent.

Accordingly, another aspect of the invention pertains to antibodies directed against a polypeptide of the invention. The term “antibody” as used herein refers to immunoglobulin molecules and immunologically active portions of immunoglobulin molecules, i.e., molecules that contain an antigen binding site which specifically binds an antigen, such as a polypeptide of the invention, i.e., an epitope of a polypeptide of the invention. A molecule which specifically binds to a given polypeptide of the invention is a molecule which binds the polypeptide, but does not substantially bind other molecules in a sample, i.e., a biological sample, which naturally contains the polypeptide. Examples of immunologically active portions of immunoglobulin molecules include F(ab) and F(ab′)2 fragments which can be generated by treating the antibody with a protein such as pepsin. The invention provides polyclonal and monoclonal antibodies. The term “monoclonal antibody” or “monoclonal antibody composition,” as used herein, refers to a population of antibody molecules that contain only one species of an antigen binding site capable of immunoreacting with a particular epitope.

Polyclonal antibodies can be prepared as described above by immunizing a suitable subject with a polypeptide of the invention as an immunogen. Preferred polyclonal antibody compositions are ones that have been selected for antibodies directed against a phage protein or protein peptide fragments of the invention. Particularly preferred polyclonal antibody preparations are ones that contain only antibodies directed against a polypeptide or polypeptides of the invention. Particularly preferred immunogen compositions are those that contain no other human proteins such as, for example, immunogen compositions made using a nonhuman host cell for recombinant expression of a polypeptide of the invention. In such a manner, the only human epitope or epitopes recognized by the resulting antibody compositions raised against this immunogen will be present as part of a polypeptide or polypeptides of the invention

All isozymes, variants or analogs of the bacteria-associated phage proteins and peptides and peptide fragments of the invention, whether natural or modified, are encompassed and included within the scope of the invention.

Methods of Treatment

The present invention features the use of the bacteria-associated lytic and holin proteins, or peptides and peptide fragments thereof in the therapeutic compositions and methods disclosed for the treatment of bacterial diseases. These proteins used are derived from a variety of bacterial species and subspecies. Examples of bacteria that cause infectious diseases which can be treated by use of the enzyme polypeptides and peptides include but are not limited, Streptococcal pygenes, Hemophilus influenza, Pseudomonas, Streptococcus pneumoniae, Streptococcus fasciae, Streptococcus group B, Listeria, Salmonella, E. coli, Campylobacter, Mycobacteria tuberculosis and Staphylococcus.

Prophylactic Methods

In one aspect, the invention provides a method for preventing in a subject, a disease or condition associated with an aberrant expression or activity of a protein or protein peptide fragments of the invention, by administering to the subject an agent which modulates expression or at least one activity of the protein or protein peptide fragments. Administration of a prophylactic agent can occur prior to the manifestation of symptoms characteristic of the aberrancy, such that a disease or disorder is prevented or, alternatively, delayed in its progression. Depending on the type of aberrancy, for example, an agonist or antagonist agent can be used for treating the subject. The appropriate agent can be determined based on screening assays described herein.

Methods of Detection

The invention also encompasses kits for detecting the presence of a polypeptide or nucleic acid of the invention in a biological sample (a test sample). Such kits can be used to determine if a subject is suffering from or is at increased risk of developing a disorder associated with aberrant expression of a polypeptide of the invention (i.e., a bacterial-related disease or disorder). For example, the kit can comprise a labeled compound or agent capable of detecting the polypeptide or mRNA encoding the polypeptide in a biological sample and means for determining the amount of the polypeptide or mRNA in the sample (i.e., an antibody which binds the polypeptide or an oligonucleotide probe which binds to DNA or mRNA encoding the polypeptide). Kits may also include instructions for observing that the tested subject is suffering from or is at risk of developing a disorder associated with aberrant expression of the polypeptide if the amount of the polypeptide or mRNA encoding the polypeptide is above or below a normal level.

For antibody-based kits, the kit may comprise, for example: (1) a first antibody (i.e., attached to a solid support) which binds to a polypeptide of the invention; and, optionally, (2) a second, different antibody which binds to either the polypeptide or the first antibody and is conjugated to a detectable agent.

For oligonucleotide-based kits, the kit may comprise, for example: (1) an oligonucleotide, i.e., a detectably labeled oligonucleotide, which hybridizes to a nucleic acid sequence encoding a polypeptide of the invention or (2) a pair of primers useful for amplifying a nucleic acid molecule encoding a polypeptide of the invention. The kit may also comprise, i.e., a buffering agent, a preservative, or a protein stabilizing agent. The kit may also comprise components necessary for detecting the detectable agent (i.e., a protein or a substrate). The kit may also contain a control sample or a series of control samples which can be assayed and compared to the test sample contained. Each component of the kit is usually enclosed within an individual container and all of the various containers are within a single package along with instructions, typically a manual, for observing whether the tested subject is suffering from or is at risk of developing a disorder associated with aberrant expression of the polypeptide.

The DNA coding of these phages and other phages may be altered to allow a recombinant enzyme to attack one cell wall at more than two locations, to allow the recombinant enzyme to cleave the cell wall of more than one species of bacteria, to allow the recombinant enzyme to attack other bacteria, or any combinations thereof. The type and number of alterations to a recombinant bacteriophage produced enzyme are incalculable.

### PHARMACEUTICAL USAGE OF PHAGE ASSOCIATED LYTIC ENZYMES

The method for treating bacterial infections comprises treating the infection with a therapeutic agent comprising an effective amount of at least one lytic enzyme genetically coded for a bacteriophage specific for the bacteria. The lytic enzyme is preferably in an environment having a pH which allows for activity of said lytic enzyme.

The lytic enzyme can be used for the treatment or prevention of Hemophilus influenza, Pseudomonas, Streptococcus pneumoniae, Streptococcus fasciae, Streptococcus group B, Listeria, Salmonella, E. coli, Campylobacter, and other bacteria, and any combination thereof.

For example, if there is a bacterial infection of the upper respiratory tract, the infection can be prophylactically or therapeutically treated with a composition comprising an effective amount of at least one lytic enzyme produced by a bacteria being infected with a bacteriophage specific for that bacteria, and a carrier for delivering the lytic enzyme to a mouth, throat, or nasal passage. It is preferred that the lytic enzyme is in an environment having a pH which allows for activity of the lytic enzyme. If an individual has been exposed to someone with the upper respiratory disorder, the lytic enzyme will reside in the mucosal lining and prevent any colonization of the infecting bacteria.

The lytic enzyme is preferably a chimeric and/or shuffled lytic enzyme which may be used in conjunction with a holin enzyme or modified or unmodified phage associated lytic enzyme. It is also preferred that the lytic enzyme is in an environment having a pH which allows for activity of the lytic enzyme. If an individual has been exposed to someone with the upper respiratory disorder, the lytic enzyme will reside in the mucosal lining and prevent any colonization of the infecting bacteria.

Two examples of bacteria which infect the upper respiratory system are Streptococcus pneumoniae and Hemophilus influenzae. In recent years, there has been an increase in the number of people, particularly children and the elderly, that are infected with or are carriers of penicillin resistant Streptococcus pneumoniae and Hemophilus. While these bacteria are normally harmless residents of the host, they are opportunistic organisms that are able to cause infections when the resistance of the host has been compromised. By eliminating or reducing the number of these organisms in the upper respiratory tract, there will be a commensurate reduction in the number of infections by these bacteria.

Infection of the Hemophilus bacteria by Bacteriophage HP1 (a member of the P2-like phage family with strong similarities to coliphages P2 and 186, and some similarity to the retronphage Ec67) produces a lytic enzyme capable of lysing the bacteria. The lytic enzyme for Streptococcus pneumoniae, previously identified as a—acetyl-muramoyl-L-alanine amidase, is produced by the infecting Streptococcus pneumoniae with the Pal bacteriophage. The therapeutic agent can contain either or both of the lytic enzymes produced by these two bacteria, and may contain other lytic enzymes for other bacteria. The composition which may be used for the prophylactic and therapeutic treatment of a strep infection includes the lysin enzyme and a means of application, (such as a carrier system or an oral delivery mode), to the mucosal lining of the oral and nasal cavity, such that the enzyme is put in the carrier system or oral delivery mode to reach the mucosal lining. Another infection which can be treated prophylactically is Streptococcus group A, which can produce what is commonly known as “strep” throat. When group C Streptococci are infected with a C1 bacteriophage, a lysin enzyme is produced specific for the lysing of Streptococcus group A.

While an “unmodified” phage associated lytic enzymes may be used for treatment of the Streptococcus, it is preferred that a shuffled or chimeric lytic enzyme be used, possibly with a holin protein.

Prior to, or at the time the lysin enzyme is put in the carrier system or oral delivery mode, it is preferred that the enzyme be in a stabilizing buffer environment for maintaining a pH range between about 4.0 and about 9.0, more preferably between about 5.5 and about 7.5 and most preferably at about 6.1.

The stabilizing buffer should allow for the optimum activity of the lysin enzyme. The buffer may be a reducing reagent, such as dithiothreitol. The stabilizing buffer may also be or include a metal chelating reagent, such as ethylenediaminetetraacetic acid disodium salt, or it may also contain a phosphate or citrate-phosphate buffer.

Means of application include, but are not limited to direct, indirect, carrier and special means or any combination of means. Direct application of the enzyme may be by nasal sprays, nasal drops, nasal ointments, nasal washes, nasal injections, nasal packings, bronchial sprays and inhalers, or indirectly through use of throat lozenges, or through use of mouthwashes or gargles, or through the use of ointments applied to the nasal nares, the bridge of the nose, or the face or any combination of these and similar methods of application. The forms in which the lysin enzyme may be administered include but are not limited to lozenges, troches, candies, injectants, chewing gums, tablets, powders, sprays, liquids, ointments, and aerosols.

The lozenge, tablet, or gum into which the enzymes are added may contain sugar, corn syrup, a variety of dyes, non-sugar sweeteners, flavorings, any binders, or combinations thereof. Similarly, any gum based products may contain acacia, carnauba wax, citric acid, corn starch, food colorings, flavorings, non-sugar sweeteners, gelatin, glucose, glycerin, gum base, shellac, sodium saccharin, sugar, water, white wax, cellulose, other binders, and combinations thereof.

Lozenges may further contain sucrose, corn starch, acacia, gum tragacanth, anethole, linseed, oleoresin, mineral oil, and cellulose, other binders, and combinations thereof. In another embodiment of the invention, sugar substitutes are used in place of dextrose, sucrose, or other sugars.

The enzyme may also be placed in a nasal spray, wherein the nasal spray is the carrier. The nasal spray can be a long acting or timed release spray, and can be manufactured by means well known in the art. An inhalant may also be used, so that the phage enzyme may reach further down into the bronchial tract, including into the lungs.

Any of the carriers for the lytic enzymes may be manufactured by conventional means. However, it is preferred that any mouthwash or similar type products not contain alcohol to prevent denaturing of the enzyme. Similarly, when the lytic enzymes are being placed in a cough drop, gum, candy or lozenge during the manufacturing process, such placement should be made prior to the hardening of the lozenge or candy but after the cough drop or candy has cooled somewhat, to avoid heat denaturation of the enzyme.

The enzyme may be added to these substances in a liquid form or in a lyophilized state, whereupon it will be solubilized when it meets body fluids such as saliva. The enzyme may also be in a micelle or liposome.

The effective dosage rates or amounts of the lytic enzyme(s) to treat the infection will depend in part on whether the lytic enzyme will be used therapeutically or prophylactically, the duration of exposure of the recipient to the infectious bacteria, the size and weight of the individual, etc. The duration for use of the composition containing the enzyme also depends on whether the use is for prophylactic purposes, wherein the use may be hourly, daily or weekly, for a short time period, or whether the use will be for therapeutic purposes wherein a more intensive regimen of the use of the composition may be needed, such that usage may last for hours, days or weeks, and/or on a daily basis, or at timed intervals during the day. Any dosage form employed should provide for a minimum number of units for a minimum amount of time. The concentration of the active units of enzyme(s) believed to provide for an effective amount or dosage of enzyme may be in the range of about 100 units/ml to about 100,000 units/ml of fluid in the wet or damp environment of the nasal and oral passages, and possibly in the range of about 100 units/ml to about 10,000 units/ml. More specifically, time exposure to the active enzyme units may influence the desired concentration of active enzyme units per ml. It should be noted that carriers that are classified as “long” or “slow” release carriers (such as, for example, certain nasal sprays or lozenges) could possess or provide a lower concentration of active (enzyme) units per ml, but over a longer period of time, whereas a “short” or “fast” release carrier (such as, for example, a gargle) could possess or provide a high concentration of active (enzyme) units per ml, but over a shorter period of time. The amount of active units per ml and the duration of time of exposure depends on the nature of infection, whether treatment is to be prophylactic or therapeutic, and other variables.

While this treatment may be used in any mammalian species, the preferred use of this product is for a human.

This composition and method may also be used for the treatment of Streptococcus A infections of the respiratory tract. When using this composition for a Streptococcus A infection, the chimeric and/or shuffled lytic enzymes should be used for the prophylactic prevention of Streptococcus infections. Similarly, in another embodiment of the invention, this method may be used for the therapeutic and, preferably, the prophylactic treatment of tuberculosis. In a preferred embodiment of the invention, the phage associated lysing enzyme for Mycobacteria tuberculosis is placed in a carrier in an inhaler. The carrier may be sterile water or a water base, or any other carrier used in an inhaler for dispersing drugs into the bronchial tract. The phage associated chimeric and/or shuffled lytic enzyme specific for tuberculosis is subject to the same conditions as the phage associated lytic enzyme for other lytic enzymes. Specifically, prior to, or at the time the enzyme is put in the carrier system or oral delivery mode, it is preferred that the enzyme be in a stabilizing buffer environment for maintaining a pH range between about 4.0 and about 9.0.

The stabilizing buffer should allow for the optimum activity of the lytic enzyme. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer.

For the prophylactic and therapeutic treatment of tuberculosis, the phage associated chimeric and/or shuffled lytic enzymes associated with tuberculosis may also be applied by direct, indirect, carriers and special means or any combination of means. Direct application of the lytic enzyme may be by nasal sprays, nasal drops, nasal ointments, nasal washes, nasal injections, nasal packings, bronchial sprays and inhalers, or indirectly through use of throat lozenges, or through use of mouthwashes or gargles, or through the use of ointments applied to the nasal nares, the bridge of the nose, or the face or any combination of these and similar methods of application. The forms in which the lytic enzyme may be administered include but are not limited to lozenges, troches, candies, injectants, chewing gums, tablets, powders, sprays, liquids, ointments, and aerosols. For the therapeutic treatment of tuberculosis, the bronchial sprays and aerosols are most beneficial, as these carriers, or means of distributing the composition, allow the lytic enzyme to reach the bronchial tubes and the lungs. An appropriate transport carrier may be attached to the enzyme to transport the enzyme across the cell membrane to the site of the bacteria. The chimeric and/or shuffled lytic enzymes may be used in combination with other chimeric and shuffled lytic enzymes, holin enzymes, other lytic enzymes, and other phage associated lytic enzymes which have not been modified or which are not “recombinant.”

Another use of a lytic enzyme is for the treatment of bacterial infections of the digestive tract. The method for treating a bacterial infection of the digestive tract comprises treating the bacterial infection with a composition comprising an effective amount of at least one lytic enzyme produced by a bacteria infected with a bacteriophage specific for the bacteria, and a carrier for delivering said lytic enzyme to the digestive tract. In a preferred embodiment of the invention, the bacterial infections being treated are selected from the group consisting of H., pyogenes, Listeria, Salmonella, E. coli, and Campylobacter. However, this method and composition will effectively treat other bacteria, when the appropriate lytic enzyme is used. The lytic enzymes used in the digestive tract may be either supplemented by chimeric and/or shuffled lytic enzymes, or may be themselves chimeric and/or shuffled lytic enzymes. Similarly, a holin enzyme may be included, which may also be a chimeric and/or shuffled lytic enzyme. The enzyme itself may be produced by recombinant methods.

In a preferred embodiment of the invention, the carrier is selected from the group consisting of suppository enemas, syrups, or enteric coated pills. These proposed carriers can be made by conventional methods. However, the only difference in their manufacture is that the enzyme being placed in the carrier must not be allowed to denature. The enzyme should be incorporated into a carrier which does not contain alcohol, and which has been cooled to a temperature that will not cause the denaturing of the enzyme. The enzyme may be incorporated in a lyophilized state, or may be incorporated in a liposome before being placed in the suppository, syrup or enteric coated pill. The enzyme placed in the composition or carrier should be in an environment having a pH which allows for activity of the lytic enzyme. To this end, the pH of the composition is preferably kept in a range of between about 2 and about 11, more preferably in a range of between about between about 4.0 and about 9.0, and even more preferably at a pH range of between about 5.5 and about 7.5. As described above with the other lytic enzyme, the pH can be moderated by the use of a buffer. The buffer may contain a reducing agent, and more specifically dithiothreitol. The buffer may also be a metal chelating reagent, such as ethylenediaminetetracetic disodium salt or the buffer may contain a citrate-phosphate buffer. As with all compositions described in this patent, the composition may, further include a bactericidal or bacteriostatic agent as a preservative.

The lytic enzyme(s) preferably are present in a concentration of about 100 to about 500,000 active enzyme units per milliliter of fluid in the wet environment of the gastrointestinal tract, preferably about 100 to about 100,000 active enzyme units per milliliter of fluid, and preferably present in a concentration of about 100 to about 10,000 active enzyme units per milliliter of fluid in the wet environment of the gastrointestinal tract.

The suppository is known in the art, and is made of glycerin, fatty acids, and similar type substances that dissolve at body temperature. As the suppository dissolves, the phage associated lytic enzyme will be released.

Another composition and use of the lytic enzyme is for the therapeutic or prophylactic treatment of bacterial infections of bums and wounds of the skin. The composition comprises an effective amount of at least one lytic enzyme produced by a bacteria infected with a bacteriophage specific for the bacteria and a carrier for delivering at least one lytic enzyme to the wounded skin. The lytic enzyme(s) used for the topical treatment of bums may be either supplemented by chimeric and/or shuffled lytic enzymes, or may themselves be chimeric and/or shuffled lytic enzymes. Similarly, a holin enzyme may be included, which may also be a chimeric and/or shuffled lytic enzyme. The mode of application for the lytic enzyme includes a number of different types and combinations of carriers which include, but are not limited to an aqueous liquid, an alcohol base liquid, a water soluble gel, a lotion, an ointment, a nonaqueous liquid base, a mineral oil base, a blend of mineral oil and petrolatum, lanolin, liposomes, protein carriers such as serum albumin or gelatin, powdered cellulose carmel, and combinations thereof. A mode of delivery of the carrier containing the therapeutic agent includes but is not limited to a smear, spray, a time-release patch, a liquid absorbed wipe, and combinations thereof. The lytic enzyme may be applied to a bandage either directly or in one of the other carriers. The bandages may be sold damp or dry, wherein the enzyme is in a lyophilized form on the bandage. This method of application is most effective for the treatment of burns.

The carriers of the compositions of the present invention may comprise semisolid and gel-like vehicles that include a polymer thickener, water, preservatives, active surfactants or emulsifiers, antioxidants, sun screens, and a solvent or mixed solvent system. U.S. Pat. No. 5,863,560 (Osborne) discusses a number of different carrier combinations which can aid in the exposure of the skin to a medicament.

Polymer thickeners that may be used include those known to one skilled in the art, such as hydrophilic and hydroalcoholic gelling agents frequently used in the cosmetic and pharmaceutical industries. Preferably, the hydrophilic or hydroalcoholic gelling agent comprises “CARBOPOL.RTM.” (B. F. Goodrich, Cleveland, Ohio), “HYPAN.RTM.” (Kingston Technologies, Dayton, N.J.), “NATROSOL.RTM.” (Aqualon, Wilmington, Del.), “KLUCEL.RTM.” (Aqualon, Wilmington, Del.), or “STABILEZE.RTM.” (ISP Technologies, Wayne, N.J.). Preferably, the gelling agent comprises between about 0.2% to about 4% by weight of the composition. More particularly, the preferred compositional weight percent range for “CARBOPOL.RTM.” is between about 0.5% to about 2%, while the preferred weight percent range for “NATROSOL.RTM.” and “KLUCEL.RTM.” is between about 0.5% to about 4%. The preferred compositional weight percent range for both “HYPAN.RTM.” and “STABILEZE.RTM.” is between about 0.5% to about 4%. CARBOPOL.RTM.” is one of numerous cross-linked acrylic acid polymers that are given the general adopted name carbomer. These polymers dissolve in water and form a clear or slightly hazy gel upon neutralization with a caustic material such as sodium hydroxide, potassium hydroxide, triethanolamine, or other amine bases. “KLUCEL.RTM.” is a cellulose polymer that is dispersed in water and forms a uniform gel upon complete hydration. Other preferred gelling polymers include hydroxyethylcellulose, cellulose gum, MVE/MA decadiene crosspolymer, PVM/MA copolymer, or a combination thereof.

Preservatives may also be used in this invention and preferably comprise about 0.05% to 0.5% by weight of the total composition. The use of preservatives assures that if the product is microbially contaminated, the formulation will prevent or diminish microorganism growth. Some preservatives useful in this invention include methylparaben, propylparaben, butylparaben, chloroxylenol, sodium benzoate, DMDM Hydantoin, 3-Iodo-2-Propylbutyl carbamate, potassium sorbate, chlorhexidine digluconate, or a combination thereof.

Titanium dioxide may be used as a sunscreen to serve as prophylaxis against photosensitization. Alternative sun screens include methyl cinnamate. Moreover, BHA may be used as an antioxidant, as well as to protect ethoxydiglycol and/or dapsone from discoloration due to oxidation. An alternate antioxidant is BHT.

Pharmaceuticals for use in all embodiments of the invention include antimicrobial agents, anti-inflammatory agents, antiviral agents, local anesthetic agents, corticosteroids, destructive therapy agents, antifungals, and antiandrogens. In the treatment of acne, active pharmaceuticals that may be used include antimicrobial agents, especially those having anti-inflammatory properties such as dapsone, erythromycin, minocycline, tetracycline, clindamycin, and other antimicrobials. The preferred weight percentages for the antimicrobials are 0.5% to 10%. Local anesthetics include tetracaine, tetracaine hydrochloride, lidocaine, lidocaine hydrochloride, dyclonine, dyclonine hydrochloride, dimethisoquin hydrochloride, dibucaine, dibucaine hydrochloride, butambenpicrate, and pramoxine hydrochloride. A preferred concentration for local anesthetics is about 0.025% to 5% by weight of the total composition. Anesthetics such as benzocaine may also be used at a preferred concentration of about 2% to 25% by weight.

Corticosteroids that may be used include betamethasone dipropionate, fluocinolone actinide, betamethasone valerate, triamcinolone actinide, clobetasol propionate, desoximetasone, diflorasone diacetate, amcinonide, flurandrenolide, hydrocortisone valerate, hydrocortisone butyrate, and desonide at concentrations of about 0.01% to about 1.0% by weight. Preferred concentrations for corticosteroids such as hydrocortisone or methylprednisolone acetate are from about 0.2% to about 5.0% by weight.

Destructive therapy agents such as salicylic acid or lactic acid may also be used. A concentration of about 2% to about 40% by weight is preferred. Cantharidin is preferably utilized in a concentration of about 5% to about 30% by weight. Typical antifungals that may be used in this invention and their preferred weight concentrations include: oxiconazole nitrate (0.1% to 5.0%), ciclopirox olamine (0.1% to 5.0%), ketoconazole (0.1% to 5.0%), miconazole nitrate (0.1% to 5.0%), and butoconazole nitrate (0.1% to 5.0%). For the topical treatment of seborrheic dermatitis, hirsutism, acne, and alopecia, the active pharmaceutical may include an antiandrogen such as flutamide or finasteride in preferred weight percentages of about 0.5% to 10%.

Typically, treatments using a combination of drugs include antibiotics in combination with local anesthetics such as polymycin B sulfate and neomycin sulfate in combination with tetracaine for topical antibiotic gels to provide prophylaxis against infection and relief of pain. Another example is the use of minoxidil in combination with a corticosteroid such as betamethasone diproprionate for the treatment of alopecia ereata. The combination of an anti-inflammatory such as cortisone with an antifungal such as ketoconazole for the treatment of tinea infections is also an example.

In one embodiment, the invention comprises a dermatological composition having about 0.5% to 10% carbomer and about 0.5% to 10% of a pharmaceutical that exists in both a dissolved state and a micro particulate state. The dissolved pharmaceutical has the capacity to cross the stratum corneum, whereas the micro particulate pharmaceutical does not. Addition of an amine base, potassium, hydroxide solution, or sodium hydroxide solution completes the formation of the gel. More particularly, the pharmaceutical may include dapsone, an antimicrobial agent having anti-inflammatory properties. A preferred ratio of micro particulate to dissolved dapsone is five or less.

In another embodiment, the invention comprises about 1% carbomer, about 80-90% water, about 10% ethoxydiglycol, about 0.2% methylparaben, about 0.3% to 3.0% dapsone including both micro particulate dapsone and dissolved dapsone, and about 2% caustic material. More particularly, the carbomer may include “CARBOPOL.RTM. 980” and the caustic material may include sodium hydroxide solution.

In a preferred embodiment, the composition comprises dapsone and ethoxydiglycol, which allows for an optimized ratio of micro particulate drug to dissolved drug. This ratio determines the amount of drug delivered, compared to the amount of drug retained in or above the stratum comeum to function in the supracorneum domain. The system of dapsone and ethoxydiglycol may include purified water combined with “CARBOPOL.RTM.” gelling polymer, methylparaben, propylparaben, titanium dioxide, BHA, and a caustic material to neutralize the “CARBOPOL.RTM.”

Any of the carriers for the lytic enzyme may be manufactured by conventional means. However, if alcohol is used in the carrier, the enzyme should be in a micelle, liposome, or a “reverse” liposome, to prevent denaturing of the enzyme. Similarly, when the lytic enzyme is being placed in the carrier, and the carrier is, or has been heated, such placement should be made after the carrier has cooled somewhat, to avoid heat denaturation of the enzyme. In a preferred embodiment of the invention, the carrier is sterile.

The enzyme may be added to these substances in a liquid form or in a lyophilized state, whereupon it will be solubilized when it meets a liquid body.

The effective dosage rates or amounts of the lytic enzyme to treat the infection, and the duration of treatment will depend in part on the seriousness of the infection, the duration of exposure of the recipient to the infectious bacteria, the number of square centimeters of skin or tissue which are infected, the depth of the infection, the seriousness of the infection, and a variety of a number of other variables. The composition may be applied anywhere from once to several times a day, and may be applied for a short or long term period. The usage may last for days or weeks. Any dosage form employed should provide for a minimum number of units for a minimum amount of time. The concentration of the active units of enzyme believed to provide for an effective amount or dosage of enzyme may be in the range of about 100 units/ml to about 500,000 units/ml of composition, preferably in the range of about 1000 units/ml to about 100,000 units/ml, and most preferably from about 10,000 to 100,000 units/ml. The amount of active units per ml and the duration of time of exposure depends on the nature of the infection, and the amount of contact the carrier allows the lytic enzyme(s) to have. It is to be remembered that the enzyme works best when in a fluid environment. Hence, effectiveness of the enzyme(s) is in part related to the amount of moisture trapped by the carrier. In another preferred embodiment, a mild surfactant is present in an amount effective to potentiate the therapeutic effect of the lytic enzyme. Suitable mild surfactants include, inter alia, esters of polyoxyethylene sorbitan and fatty acids (Tween series), octylphenoxy polyethoxy ethanol (Triton-X series), n-Octyl-.beta.-D-glucopyranoside, n-Octyl-.beta.-D-thioglucopyranoside, n-Decyl-.beta.-D-glucopyranoside, n-Dodecyl-.beta.-D-glucopyranoside, and biologically occuring surfactants, e.g., fatty acids, glycerides, monoglycerides, deoxycholate and esters of deoxycholate.

In order to accelerate treatment of the infection, the therapeutic agent may further include at least one complementary agent which can also potentiate the bactericidal activity of the lytic enzyme. The complementary agent can be penicillin, synthetic penicillins bacitracin, methicillin, cephalosporin, polymyxin, cefaclor. Cefadroxil, cefamandole nafate, cefazolin, cefixime, cefmetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam , cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef. mafate, chelating agents, streptomycin, erythromycin, chloramphenicol, numerous other antibiotics, and any combinations thereof in amounts which are effective to synergistically enhance the therapeutic effect of the lytic enzyme. It should be noted that virtually any antibiotic may be used as complementary agents for or with any use of the recombinant lytic enzymes.

Additionally, the therapeutic agent may further comprise the enzyme lysostaphin for the treatment of any Staphylococcus aureus bacteria. Mucolytic peptides, such as lysostaphin, have been suggested to be efficacious in the treatment of S. aureus infections of humans (Schaffner et al., Yale J. Biol. & Med., 39:230 (1967) and bovine mastitis caused by S. aureus (Sears et al., J. Dairy Science, 71 (Suppl. 1): 244(1988)) Lysostaphin, a gene product of Staphylococcus simulans, exerts a bacteriostatic acid bactericidal effect upon S. aureus by enzymatically degrading the polyglycine crosslinks of the cell wall (Browder et al., Res. Comm., 19: 393-400 (1965)). U.S. Pat. No. 3,278,378 describes fermentation methods for producing lysostaphin from culture media of S. staphylolyticus, later renamed S. simulans. Other methods for producing lysostaphin are further described in U.S. Pat. Nos. 3,398,056 and 3,594,284. The gene for lysostaphin has subsequently been cloned and sequenced (Recsei et al., Proc. Natl. Acad. Sci. USA, 84: 1127-1131 (1987)). The recombinant mucolytic bactericidal protein, such as r-lysostaphin, can potentially circumvent problems associated with current antibiotic therapy because of its targeted specificity, low toxicity and possible reduction of biologically active residues. Furthermore, lysostaphin is also active against non-dividing cells, while most antibiotics require actively dividing cells to mediate their effects (Dixon et al., Yale J. Biology and Medicine, 41: 62-68 (1968)). Lysostaphin, in combination with the lysin enzyme, can be used in the presence or absence of the listed antibiotics. There is a degree of added importance in using both lysostaphin and the lysin enzyme in the same therapeutic agent. Frequently, when a body has a bacterial infection, the infection by one genus of bacteria weakens the body or changes the bacterial flora of the body, allowing other potentially pathogenic bacteria to infect the body. One of the bacteria that sometimes co-infects a body is Staphylococcus aureus. Many strains of Staphylococcus aureus produce penicillinase, such that Staphylococcus, Streptococcus, and other gram positive bacterial strains will not be killed by standard antibiotics. Consequently, the use of the lysin and lysostaphin, possibly in combination with antibiotics, can serve as the most rapid and effective treatment of bacterial infections. In yet another preferred embodiment, the invention may include mutanolysin, and lysozyme

In preferred embodiments of the invention, the chimeric and/or shuffled lytic enzymes for Pseudomonas, Staphylococcus, and Streptococcus, jointly or individually, may be incorporated into the carrier, or into a bandage to be used on burn patients, or in a solution or cream carrier. These enzymes may be used in combination with holin proteins and other lytic enzymes.

Yet another use of-lytic enzymes is for the prophylactic or therapeutic treatment of vaginal infections. This treatment comprises treating the vaginal infection with an effective amount of at least one lytic enzyme produced by a bacteria being infected with a bacteriophage specific for that bacteria, wherein that lytic enzyme is incorporated in a carrier to be placed in a vagina. The lytic enzyme(s) used to treat bacterial infections of the vagina may be either supplemented by chimeric and/or shuffled lytic enzymes, or may be itself a chimeric and/or shuffled lytic enzyme. Similarly, a holin enzyme may be included, which may also be a chimeric and/or shuffled lytic enzyme. The preferred carrier is a tampon, or vaginal douche. A pad may also be used as a carrier, although it is not as effective. While any number of bacteria could be treated using this composition and method, it is believed that the most optimum use of this treatment composition and method would be for the treatment of an E. coli and Streptococcus B infection. Vaginal infections caused by Group B Streptococcus can cause neonatal meningitis resulting in brain damage and premature death. Lytic enzymes incorporated into tampon specific for group B Strep would eliminate the group B organisms without disturbing normal floraso that woman would not be overcome by yeast infection post antibiotic therapy. The use of the lytic enzymes in the vagina would best provide a prophylactic effect, although therapeutic use would also be advisable.

To produce a pad or tampon containing the enzyme, the lytic enzymes can be applied in a solution to the tampon, and allowed to dry. The lytic enzyme may be incorporated into the pad or tampon by any other means known in the art, including lyophilization, spraying, etc. The tampons and pads may also be kept slightly moist, and in a sealed wrapper until ready for use. In that case, bactericide and bacteriostatic compounds and inhibitors should be present in the tampons and pads. The method to be used for incorporating the lytic enzyme into the tampon or pad can be one of the methods known in the art for incorporating a pharmaceutical product. In another embodiment of the invention, the lytic enzyme is incorporated into a vaginal suppository. The vaginal suppository into which the lytic enzyme is being incorporated may be a standard vaginal suppository, comprised of glyceride, alginate, starch, other standard binders and any combinations thereof.

When using a tampon as the carrier, it is best to insert the tampon in the vagina and leave it in for up to 12 hours to distribute the enzyme vaginally.

As with other lytic enzymes, it is preferable that the pH be kept in a range of about 4.0 and about 9.0 even more preferably at a pH range of between about 5.5 and about 7.5. As described above with the other lytic enzyme, the pH can be moderated by the use of a buffer. The buffer may contain a reducing agent, and more specifically dithiothreitol. The buffer may also contain a metal chelating reagent, such as ethylenediaminetetracetic disodium salt or the buffer may be a citrate-phosphate buffer. As with all compositions described in this patent, the composition may, further include a bactericidal or bacteriostatic agent as a preservative.

The lytic enzyme(s) are preferably present in a concentration of about 100 to about 500,000 active enzyme units per milliliter of fluid in the wet environment of the vaginal tract, preferably about 100 to about 100,000 active enzyme units per milliliter of fluid, and preferably present in a concentration of about 100 to about 10,000 active enzyme units per milliliter of fluid in the wet environment of the vaginal tract.

Another use of the invention is for the prophylactic and therapeutic treatment of eye infections. The method of treatment comprises administering eye drops which comprise an effective amount of at least one lytic enzyme produced by the bacteria being infected with a bacteriophage specific for the bacteria and a carrier capable of being safely applied to an eye, with the carrier containing the lytic enzyme. In a preferred embodiment of the invention, the bacteria being treated is Hemophilus or Staphylococcus The eye drops are in the form of an isotonic solution. The pH of the solution should be adjusted so that there is no irritation of the eye, which in turn would lead to possibly infection by other organisms, and possibly to damage to the eye. While the pH range should be in the same range as for other lytic enzymes, the most optimal pH will be in the range of from 6.0 to 7.5. Similarly, buffers of the sort described above for the other lytic enzymes should also be used. Other antibiotics which are suitable for use in eye drops may be added to the composition containing the lytic enzymes Bactericides and bacteriostatic compounds may also be added. As stated above, this lytic enzyme may be either supplemented by chimeric and/or shuffled lytic enzymes, or may be itself a chimeric and/or shuffled lytic enzyme. Similarly, a holin enzyme may be included which may also be a chimeric and/or shuffled lytic enzyme.

It is to be remembered that all of the enzymes can be used for prophylactic and therapeutic treatments of the bacteria for which the enzymes are specific.

Additionally, a carrier may have more than one lytic enzyme. For instance, a throat lozenge may comprise just a lysin enzyme (which lyses the Streptococcus A strain causing “strep” throat) or it may also include the lytic enzymes for Hemophilus. Similarly, the carrier for treating burns and wounds, or infections of the skin, may contain just one lytic enzyme, or a combination of lytic enzymes, for the treatment of Pseudomonas, Streptococcus, Staphylococcus, or any other of a number of bacteria. The carrier may include any combination of lytic enzymes, shuffled lytic enzymes, chimeric lytic enzymes, and holin enzymes,

Lytic enzymes can also be used to fight dental caries. For example, a lytic enzyme specific for Streptococcus mutans,—may be incorporated in a toothpaste or oral wash. Similarly, this lytic enzyme may also be incorporated into a chewing gum or lozenge. Any other carrier can be used that allows for the exposure of the mouth, gums, and teeth to the lytic enzyme. Other target cariogenic or periodonto-pathogenic bacteria which may be treated come from the genera Actinobacillus, Actinomyces, Bacteroides, Capnocytophaga, Eikenella, Eubacterium, Fusobacterium, Haemophilus, Lactobacillus, Peptostreptococcus, Porphyromonas, Prevotella, Rothia, Selenomonas, Streptococcus, Treponema, and Wolinella. More specific gram positive, caries-related species include but are not limited to: Actinromyces viscosus, A. naeslundii, and Streptococcus mutans, S. sobrinus, and Lactobacillus casei.

The lytic enzyme may also be incorporated in a lyophilized or dried form in tooth powder. If the lytic enzyme is to be used in an oral wash, it is preferred that the oral wash not contain any alcohol, so as to not denature the enzyme. The enzyme can also be in a liposome when mixed in with the toothpaste or oral wash. The concentrations of the enzyme units per ml of toothpaste, oral wash, chewing gum, candy or lozenge can be in the range of from about 100 units/ml to about 500,000 units/ml of composition, preferably in the range of about 1000 units/ml to about 100,000 units/ml, and most preferably from about 10,000 to 100,000 units/ml. In some circumstances, the amounts of enzyme may range up to over 1,000,000 units/ml, and possibly much higher. The pH of the toothpaste or oral wash should be in a range that allows for the optimum performance of the enzyme, while not causing any discomfort to the user of the toothpaste or oral wash. Again, as with the other uses of lytic enzymes, the lytic enzyme used to treat dental caries may be either supplemented by chimeric and/or shuffled lytic enzymes, or may be itself a chimeric and/or shuffled lytic enzyme. Similarly, a holin enzyme may be included, which may also be a chimeric and/or shuffled lytic enzyme. The toothpastes, lozenges, gums, mouth wash, candy,and toothpowders may include any of their normal substances, as long as they do not interfere with the actions and viability of the enzyme(s). Similarly, there may be as many different specific enzymes as desired. In a candy, it is preferred that an artificial sweetener be used, although this is not always necessary.

The lytic enzymes may also be adminiiistered parenterally. The lytic enzyme, holin lytic enzyme, chimeric enzyme, shuffled enzyme, and combinations thereof may be administered parenterally using an effective amount of a therapeutic agent, the therapeutic agent comprising at least one lytic enzyme produced by a bacteria infected with a bacteriophage specific for said bacteria selected from the group consisting of holin lytic enzymes, chimeric lytic enzymes, shuffled lytic enzymes, and combinations thereof, and a carrier for delivering the lytic enzyme to the site of the infection.

The composition may be used for the therapeutic treatment of Pseudomonas, Clostridium, Staphylococcus infections, among others.

A number of different bacteria may be treated. Among the-bacteria which most often infect deep tissues, and, more specifically connective tissues, are Group A Streptococcus, Staphylococcus, Pseudomonas, and Clostridium. More than one lytic enzyme may be introduced into the infected body at a time.

A number of different methods may be used to introduce the lytic enzyme(s). These methods include introducing the lytic enzyme intravenously, intramuscularly, subcutaneously, and subdermally.

In one preferred embodiment of the invention, a deep tissue infection may be treated by injecting into the infected tissue of the patient a therapeutic agent comprising the appropriate lytic enzyme(s) (holin lytic enzyme, chimeric lytic enzyme and/or shuffled lytic enzyme) and a carrier for the enzyme. The carrier may be comprised of distilled water, a saline solution, albumin, a serum, or any combinations thereof. More specifically, solutions for infusion or injection may be prepared in a conventional manner, e.g. with the addition of preservatives such as p-hydroxybenzoates or stabilizers such as alkali metal salts of ethylene-diamine tetraacetic acid, which may then be transferred into fusion vessels, injection vials or ampules. Alternatively, the compound for injection may be lyophilized either with or without the other ingredients and be solubilized in a buffered solution or distilled water, as appropriate, at the time of use. Non-aqueous vehicles such as fixed oils and ethyl oleate are also useful herein.

In cases where intramuscular injection is the chosen mode of administration, an isotonic formulation is preferably used. Generally, additives for isotonicity can include sodium chloride, dextrose, mannitol, sorbitol and lactose. In some cases, isotonic solutions such as phosphate buffered saline are preferred. Stabilizers include gelatin and albumin. In some embodiments, a vasoconstriction agent is added to the formulation. The pharmaceutical preparations according to the present invention are provided sterile and pyrogen free.

The carrier suitably contains minor amounts of additives such as substances that enhance isotonicity and chemical stability. Such materials are nontoxic to recipients at the dosages and concentrations employed, and include buffers such as phosphate, citrate, succinate, acetic acid, and other organic acids or their salts; antioxidants such as ascorbic acid; low molecular weight (less than about ten residues) polypeptides, e.g., polyarginine or tripeptides; proteins, such as serum albumin, gelatin, or immunoglobulins; hydrophilic polymers such as polyvinylpyrrolidone; glycine; amino acids such as glutamic acid, aspartic acid, histidine, or arginine; monosaccharides, disaccharides, and other carbohydrates including cellulose or its derivatives, glucose, mannose, trehalose, or dextrins; chelating agents such as EDTA; sugar alcohols such as mannitol or sorbitol; counter-ions such as sodium; non-ionic surfactants such as polysorbates, poloxamers, or polyethylene glycol (PEG); and/or neutral salts, e.g., NaCl, KCl, MgCl.sub.2, CaCl.sub.2, etc.

Glycerin or glycerol (1,2,3-propanetriol) is commercially available for pharmaceutical use. It may be diluted in sterile water for injection, or sodium chloride injection, or other pharmaceutically acceptable aqueous injection fluid, and used in concentrations of 0.1 to 100% (v/v), preferably 1.0 to 50% and more, but preferably about 20%.

DMSO, an aprotic solvent with a remarkable ability to enhance penetration of many locally applied drugs, may be diluted in sterile water for injection, or sodium chloride injection, or other pharmaceutically acceptable aqueous injection fluid, and used in concentrations of 0.1 to 100% (v/v).

The carrier vehicle may also include Ringer's solution, a buffered solution, and dextrose solution, particularly when an intravenous solution is prepared.

Prior to, or at the time the lytic enzyme is put in the carrier system or oral delivery mode, it is preferred that the enzyme be in a stabilizing buffer environment for maintaining a pH range between about 4.0 and about 9.0, more preferably between about 5.5 and about 7.5 and most preferably at about 6.1. This is pH range is most suitable for the lysin enzyme for Streptococcus.

The stabilizing buffer should allow for the optimum activity of the lysin enzyme. The buffer may be a reducing reagent, such as dithiothreitol. The stabilizing buffer may also be or include a metal chelating reagent, such as ethylenediaminetetracetic acid disodium salt, or it may also contain a phosphate or citrate-phosphate buffer. The buffers found in the carrier can serve to stabilize the environment for the lytic enzymes.

The effective dosage rates or amounts of the chimeric and/or shuffled lytic enzymes to treat the infection, and the duration of treatment will depend in part on the seriousness of the infection, the duration of exposure of the recipient to the infectious bacteria, the number of square centimeters of skin or tissue which are infected, the depth of the infection, the seriousness of the infection, and a variety of a number of other variables. The composition may be applied anywhere from once to several times a day, and may be applied for a short or long term period. The usage may last for days or weeks. Any dosage form employed should provide for a minimum number of units for a minimum amount of time. The concentration of the active units of enzyme believed to provide for an effective amount or dosage of enzyme may be in the range of about 100 units/ml to about 500,000 units/ml of composition, preferably in the range of about 1000 units/ml to about 100,000 units/ml, and most preferably from about 10,000 to 100,000 units/ml. The amount of active units per ml and the duration of time of exposure depends on the nature of infection, and the amount of contact the carrier allows the lytic enzyme to have. It is to be remembered that the enzyme works best when in a fluid environment. Hence, effectiveness of the enzyme is in part related to the amount of moisture trapped by the carrier. For the treatment of septicemia, there should be a continuous intravenous flow of therapeutic agent into the blood stream. The concentration of lytic enzymes for the treatment of septicemia is dependent upon the seriousness of the infection.

In order to accelerate treatment of the infection, the therapeutic agent may further include at least one complementary agent which can also potentiate the bactericidal activity of the lytic enzyme. The complementary agent can be penicillin, synthetic penicillins bacitracin, methicillin, cephalosporin, polymyxin, cefaclor. Cefadroxil, cefamandole nafate, cefazolin, cefixime, cefmetazole, cefonioid, cefoperazone, ceforanide, cefotanme, cefotaxime, cefotetan, cefoxitin, cefpodoxime proxetil, ceftazidime, ceftizoxime, ceftriaxone, cefriaxone moxalactam, cefuroxime, cephalexin, cephalosporin C, cephalosporin C sodium salt, cephalothin, cephalothin sodium salt, cephapirin, cephradine, cefuroximeaxetil, dihydratecephalothin, moxalactam, loracarbef. mafate, chelating agents, streptomycin, erythromycin, chloramphenicol, numerous other antibiotics, and any combinations thereof in amounts which are effective to synergistically enhance the therapeutic effect of the lytic enzyme. As previously noted, virtually any antibiotic may be used with the the various lytic enzymes, which include the shuffled and/or chimeric lytic enzymes, the holin enzymes, etc.

Additionally, the therapeutic agent may further comprise the enzyme lysostaphin for the treatment of any Staphylococcus aureus bacteria. In yet another preferred embodiment, the invention may include mutanolysin, and lysozyme

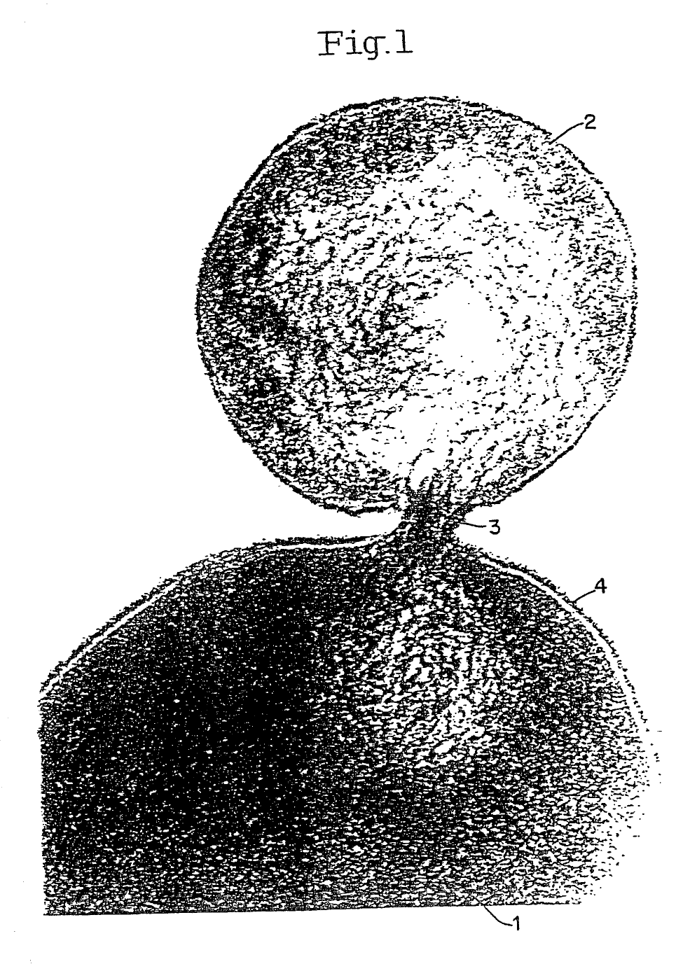

The use of lytic enzymes, including but not limited to holin lytic enzymes, chimeric lytic enzymes, shuffled lytic enzymes, and combinations thereof, rapidly lyse the bacterial cell. The thin section electron micrograph of FIG. 1 shows the results of a group A streptococci 1 treated for 15 seconds with lysin. The micrograph (25,000× magnification) shows the cell contents 2 pouring out through a hole 3 created in the cell wall 4 by the lysin enzyme.

As noted above, the use of the holin lytic enzyme, the chimeric lytic enzyme, and/or the shuffled lytic enzyme, may be accompanied by the use of a “natural” lytic enzyme, which has not been modified by the methods cited in U.S. Pat. No. 6,132,970, or by similar state of the art methods. Similarly, the natural proteins or lytic enzyme may be used without the chimeric or shuffled lytic enzymes. The phage associated lytic enzyme may be prepared as shown in the following example:

### EXAMPLE 1

Harvesting Phage Associated Lytic Enzyme

Group C streptococcal strain 26RP66 (ATCC #21597) or any other group C streptococcal strain is grown in Todd Hewitt medium at 37.degree. C. to an OD of 0.23 at 650 nm in an 18 mm tube. Group C bacteriophage (C1) (ATCC #21597-B1) at a titer of 5.times.10.sup.6 is added at a ratio of 1 part phage to 4 parts cells. The mixture is allowed to remain at 37.degree. C. for 18 min at which time the infected cells are poured over ice cubes to reduce the temperature of the solution to below 15.degree. C. The infected cells are then harvested in a refrigerated centrifuge and suspended in 1/300th of the original volume in 0.1M phosphate buffer, pH 6.1 containing 5.times.10.sup.−3 M dithiothreitol and 10 ug of DNAase. The cells will lyse releasing phage and the lysin enzyme. After centrifugation at 100,000.times. g for 5 hrs to remove most of the cell debris and phage, the enzyme solution is aliquoted and tested for its ability to lyse Group A Streptococci.

The number of units/ml in a lot of enzyme is determined to be the reciprocal of the highest dilution of enzyme required to reduce the OD650 of a suspension of group A streptococci at an OD of 0.3 to 0.15 in 15 minutes. In a typical preparation of enzyme 4.times.10.sup.5 to 4.times.10.sup.6 units are produced in a single 12 liter batch.

Use of the enzyme in an immunodiagnostic assay requires a minimum number of units of lysin enzyme per test depending on the incubation times required. The enzyme is diluted in a stabilizing buffer maintaining the appropriate conditions for stability and maximum enzymatic activity, inhibiting nonspecific reactions, and in some configurations contains specific antibodies to the Group A carbohydrate. The preferred embodiment is to use a lyophilized reagent which can be reconstituted with water. The stabilizing buffer can comprise a reducing reagent, which can be dithiothreitol in a concentration from 0.001M to 1.0M, preferably 0.005M. The stabilizing buffer can comprise an immunoglobulin or immunoglobulin fragments in a concentration of 0.001 percent to 10 percent, preferably 0.1 percent. The stabilizing buffer can comprise a citrate-phosphate buffer in a concentration from 0.001M to 1.0M, preferably 0.05M. The stabilizing buffer can have a pH value in the range from 5.0 to 9.0. The stabilizing buffer can comprise a bactericidal or bacteriostatic reagent as a preservative. Such preservative can be sodium azide in a concentration from 0.001 percent to 0.1 percent, preferably 0.02 percent.

The preparation of phage stocks for lysin production is the same procedure described above for the infection of group C streptococcus by phage in the preparation of the lysin enzyme. However, instead of pouring the infected cells over ice, the incubation at 37.degree. C. is continued for a total of 1 hour to allow lysis and release of the phage and the enzyme in the total volume. In order for the phage to be used for subsequent lysin production the residual enzyme must be inactivated or removed to prevent lysis from without of the group C cells rather than phage infection.

The use of chimeric or shuffled enzymes shows a great improvement as to the properties of the enzyme, as illustrated by the following examples:

### EXAMPLE 2

A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetrating of the proteins of either the inner membrane or the inner and outer membranes of the E. coli. FEMS Microbiol. Lett. 1998 Jul. 1, 164(1); 159-67.

Also, an active chimeric cell wall lytic enzyme (TSL) is constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumoccal cell walls.

### EXAMPLE 3

Isolation of the Pal Lytic Enzyme:

Recombinant E.coli DH5 (PMSP11) containing the pal lytic enzyme gene were grown overnight, induced with lactose, pelleted, resupended in phosphate buffer, broken by sonication. After centrifugation, the Pal enzyme in the supernatant was purified in a single step using a DEAE-cellulose colurnn and elution with choline. Protein content was analyzed with the Bradford method. Using this method, a single protein band was identified by SDS-PAGE.

### EXAMPLE 4

Killing Assay:

S. pneumoniae of various serotypes and 8 different viridans streptococi were grown overnight and for most assays diluted and re-grown for 6 h to log phase of growth, pelleted and resupended in 0.9% saline to an OD @620nm of 1.0. In some experiments, stationary phase organisms were used. Killing assays were performed by adding 100, 1,000 or 10,000 U/mL of Pal to an equal volume of the bacterial suspension and incubating for 15 minutes at 37 C. Phosphate buffer served as control in place of enzyme. Bacterial counts before and after Pal or control phosphate buffer treatment were assessed by serial 10-fold dilutions at various time points and plated to determine colony forming units.

One unit (U) of Pal was defined as the highest dilution at which Pal decreased the OD of a pneumococcal strain by half in 15 minutes.

### EXAMPLE 5

Production of Chimeric Lytic Enzymes

A number of chimeric lytic enzymes have been produced and studied. Gene E-L, a chimeric lysis constructed from bacteriophages phi X174 and MS2 lysis proteins E and L, respectively, was subjected to internal deletions to create a series of new E-L clones with altered lysis or killing properties. The lytic activities of the parental genes E, L, E-L, and the internal truncated forms of E-L were investigated in this study to characterize the different lysis mechanism, based on differences in the architecture of the different membranes spanning domains. Electron microscopy and release of marker enzymes for the cytoplasmic and periplasmic spaces revealed that two different lysis mechanisms can be distinguished depending on penetrating of the proteins of either the inner membrane or the inner and outer membranes of the E. coli. FEMS Microbiol. Lett. 1998 Jul. 1, 164(1); 159-67.

Also, an active chimeric cell wall lytic enzyme (TSL) is constructed by fusing the region coding for the N-terminal half of the lactococcal phage Tuc2009 lysin and the region coding for the C-terminal domain of the major pneumococcal autolysin. The chimeric enzyme exhibited a glycosidase activity capable of hydrolysing choline-containing pneumoccal cell walls.

### EXAMPLE 6

Isolation of the Pal Lytic Enzyme

Recombinant E.coli DH5 (pMSP11) containing the pal lytic enzyme gene were grown overnight, induced with lactose, pelleted, resupended in phosphate buffer, broken by sonication. After centrifugation, the Pal enzyme in the supernatant was purified in a single step using a DEAE-cellulose column and elution with choline. Protein content was analyzed with the Bradford method. Using this method, a single protein band was identified by SDS-PAGE.

### EXAMPLE 7

Killing Assay

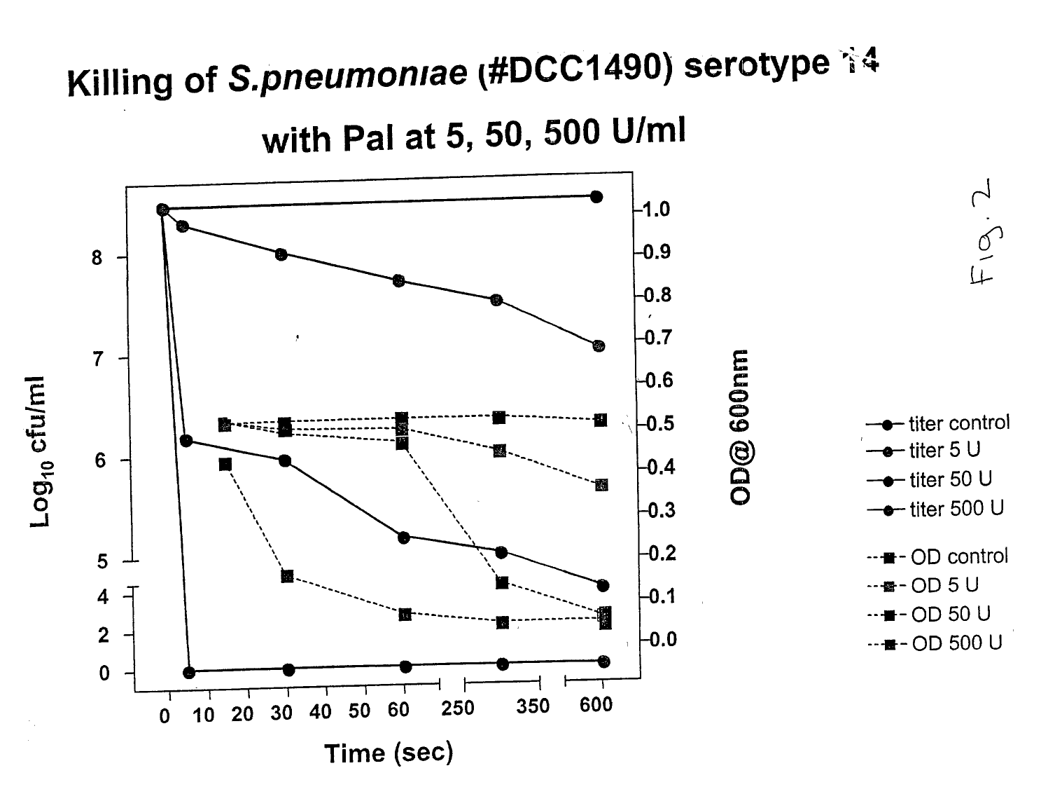

S. pneumoniae of various serotypes and 8 different viridans streptococi were grown overnight and for most assays diluted and re-grown for 6 h to log phase of growth, pelleted and resupended in 0.9% saline to an OD @620 nm of 1.0. In some experiments, stationary phase organisms were used. Killing assays were performed by adding 100, 1,000 or 10,000 U/mL of Pal to an equal volume of the bacterial suspension and incubating for 15 minutes at 37 C. Phosphate buffer served as control in place of enzyme. Bacterial counts before and after Pal or control phosphate buffer treatment were assessed by serial 10-fold dilutions at various time points and plated to determine colony forming units. One unit (U) of Pal was defined as the highest dilution at which Pal decreased the OD of a pneumococcal strain by half in 15 minutes. The results, (see FIG. 2) show that the viability of Pneumococci dropped more than 8 logs in five seconds after adding the Pal enzyme.

### EXAMPLE 8

Susceptability of Oral Streptoccocci to Pal Enzyme

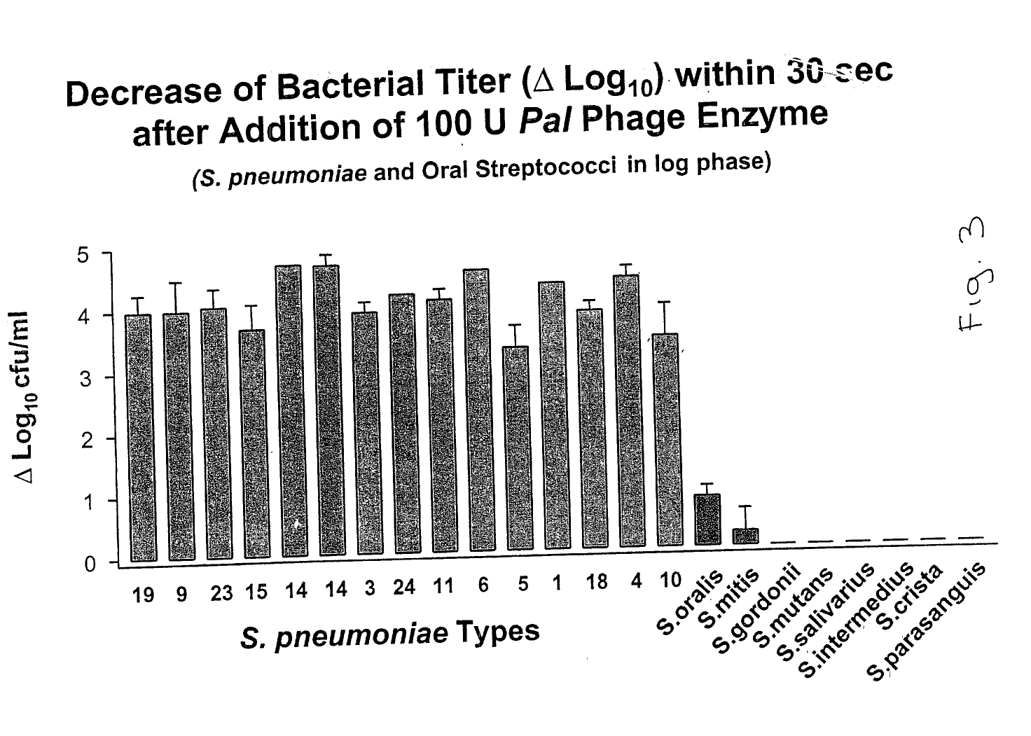

Various serotypes of oral streptoccoci were tested against bacteria-associated lytic enzymes, in particular, the Pal enzyme. A variety of S. pneumoniae type bacteria was also included in the test. Pal enzyme were used at a concentration of 100 U of the purified enzyme. As can be seen in FIG. 3 all S. pneumoniae serotypes are killed (˜4 logs) within the 30 seconds of exposure. Of the oral streptococci tested, only S. oralis and S. mitis show low sensitivity to the Pal enzyme.

### EXAMPLE 9

Susceptability of Stationary Phase bacteria to Lytic Enzyme

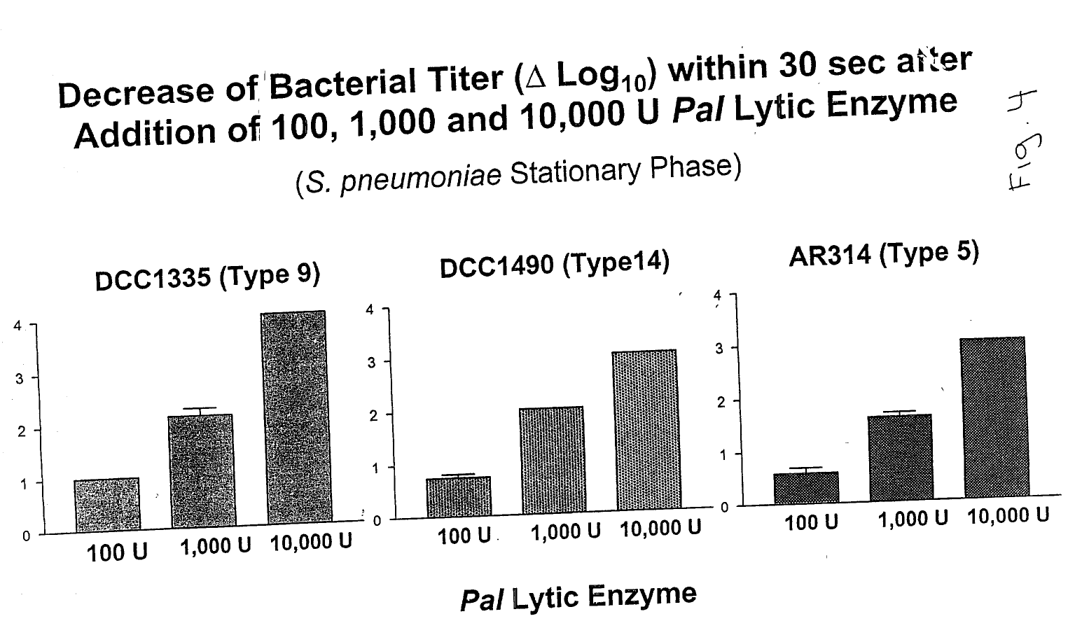

In order to confirm that activity of lytic enzymes are independent of the bacterial growth, several serotypes of serotypes of S.pneumoniae at stationary phase of growth were tested against lytic enzymes. In particular, 3 strains of Pal lytic enzyme were used against 3 sereotypes of S. pneumoniae. The results show that that all bacterial strains tested against Pal enzyme were killed in 30 seconds (see FIG. 4). An approximately 2-log drop in viability of the bacteria occurred with 1,000 U of enzyme, as opposed to about 3-4 log drop in the viability with 10,000 units.

### EXAMPLE 10

Effect of Pal Lytic Enzyme on Log-Phase and Stationary Phase Oral Streptococci.

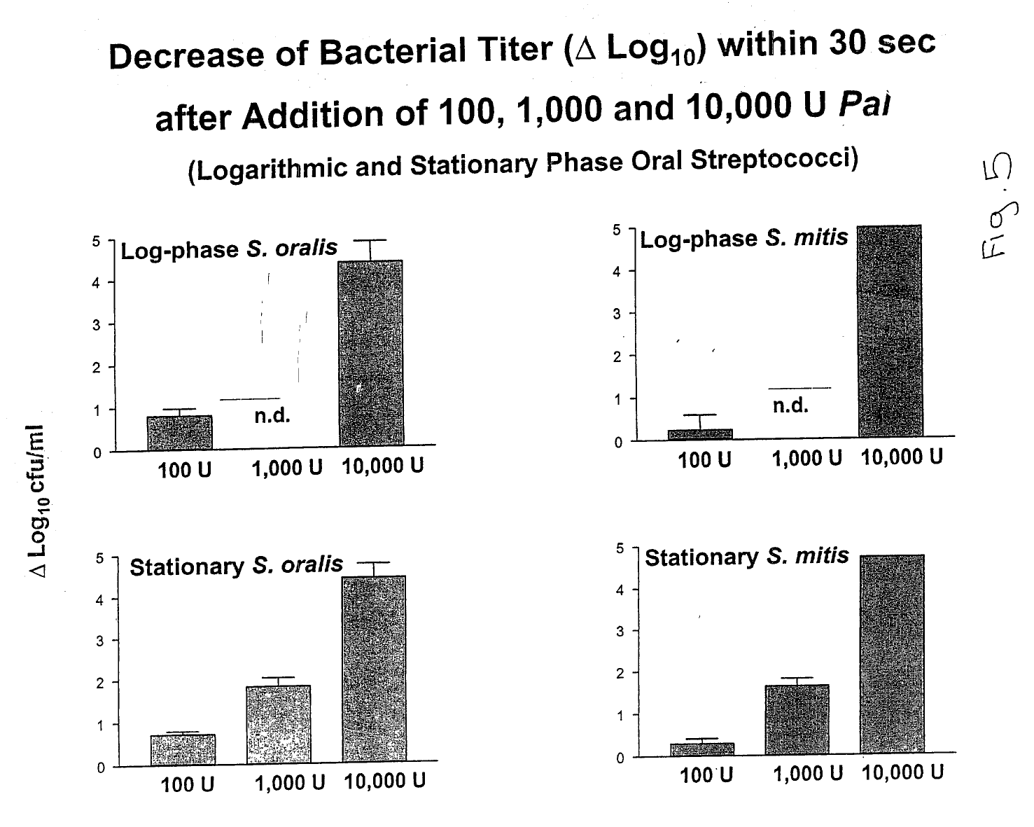

Streptococci oralis and Streptococci.mitis in log or stationary phases of growth were treated with different concentrations of the Pal lytic enzyme. Viability was measured after 30 seconds. Results, as shown in FIG. 5, indicate that both bacterial species were equally sensitive to the Pal enzyme in both log or stationary phases of growth.

In all of the uses for the enzyme, the form of the enzyme may be “natural,” formed by recombinant or “genetically engineered” means, and may be a shuffled, chimeric or otherwise altered enzyme. A holin protein may be used in any of the illnesses discussed, and more than one enzyme may be used in each composition.

It should also be noted that each publication cited herein is incorporated by reference in its entirety.

Many modifications and variations of the present invention are possible in light of the above teachings. It is, therefore, to be understood within the scope of the appended claims the invention may be protected otherwise than as specifically described.

What we claim is:

1) A lozenge for treating bacterial dental caries comprising:
(i) an effective amount of at least one lytic enzyme genetically coded for by a specific bacteriophage specific for a bacteria causing bacterial dental caries, said bacteria selected from the group consisting of Actinobacillus, Actinomyces, Bacteroides, Capnocytophaga, Eikenella, Eubacterium, Fusobacterium, Haemophilus, Lactobacillus, Peptostreptococcus, Porphyromonas, Prevotella, Rothia, Selenomonas, Streptococcus, Treponema, and Wolinella, and combinations thereof, wherein said at least one said lytic enzyme is specific for, and has the ability to digest, a cell wall of one of said bacteria, said at least one lytic enzyme being coded for by the same said bacteriophage capable of infecting said bacteria being digested wherein the lytic enzyme has been made chimeric through genetic manipulation; and
(ii).a dental carrier for delivering said enzyme to the mouth, gums, and teeth.

2) The lozenge according to claim 1, wherein said bacteria being treated is selected from the group consisting of Actinomyces viscosus, A. naeslundii, and Streptococcus mutans, S. sobrinus, Lactobacillus casei and combinations thereof.

3) The method according to claim 1, further comprising a holin protein.

4) The method according to claim 1, wherein the lytic enzyme has been made chimeric through shuffling.

5) A candy comprising:
(i) an effective amount of at least one lytic enzyme genetically coded for by a specific bacteriophage specific for a bacteria causing bacterial dental caries, said bacteria selected from the group consisting of Actinobacillus, Actinomyces, Bacteroides, Capnocytophaga, Eikenella, Eubacterium, Fusobacterium, Haemophilus, Lactobacillus, Peptostreptococcus. Porphyromonas, Prevotella, Rothia, Selenomonas, Streptococcus, Treponema, and Wolinella, and combinations thereof, wherein said at least one said lytic enzyme is specific for, and has the ability to digest, a cell wall of one of said bacteria, said at least one lytic enzyme being coded for by the same said bacteriophage capable of infecting said bacteria being digested wherein the lytic enzyme has been made chimeric through genetic manipulation; and
(ii).a dental carrier for delivering said enzyme to the mouth, gums, and teeth.

6) The candy according to claim 5, wherein said bacteria being treated is selected from the group consisting of Actinomyces viscosus, A. naeslundii, and Streptococcus mutans, S. sobrinus, Lactobacillus casei, and combinations thereof.

7) The method according to claim 5, further comprising a holin protein.

8) The method according to claim 5, wherein the lytic enzyme has been made chimeric through shuffling.
